(19) United States
(12) Patent Application Publication
(10) Pub. No.: US 2025/0092106 A1
(43) Pub. Date: Mar. 20, 2025

(54) VEGF MUTANTS AND MODULATION OF INTEGRIN-MEDIATED SIGNALING

(71) Applicant: The Regents of the University of California, Oakland, CA (US)

(72) Inventors: Yoshikazu Takada, Oakland, CA (US); Yoko Takada, Oakland, CA (US)

(21) Appl. No.: 18/832,202

(22) PCT Filed: Jan. 20, 2023
(86) PCT No.: PCT/US2023/011300

§ 371 Date: Jul. 23, 2024

Related U.S. Application Data
(60) Provisional application No. 63/302,702, filed on Jan. 25, 2022.

(51) Int. Cl. C07K 14/475; G01N 33/68

(52) U.S. Cl. CPC C07K 14/475; G01N 33/68; G01N 2333/475; G01N 2333/70546

(57) ABSTRACT

The present invention resides in the discovery that the specific interaction between vascular endothelial growth factor (VEGF) and integrin, especially integrin αvβ3, is involved in cellular signaling mediated by VEGF-integrin, such as angiogenesis and inflammatory responses. Thus, this invention provides for a novel method for inhibiting angiogenesis or inflammation induced by integrin signaling by using an inhibitor of VEGF-integrin binding, such as a dominant negative mutant of VEGF without integrin-binding capability. A method for identifying inhibitors of VEGF-integrin binding is also described. Further disclosed are polypeptides, nucleic acids, host cells, and corresponding compositions for inhibiting VEGF-integrin signaling.

### RELATED APPLICATIONS

This application claims priority to U.S. Provisional Patent Application No. 63/302,702, filed Jan. 25, 2022, the contents of which are incorporated by reference in the entirety for all purposes.

### BACKGROUND OF THE INVENTION

Each year, nearly 10 million cancer deaths are recorded worldwide, or about 26,000 per day and 1 in every 6 deaths. In the US, annual deaths are about 600,000 from various forms of cancer. Angiogenesis, or the process of new blood vessel formation, is critical for the progression and metastasis of all solid forms of cancer, since tumor mass depends on blood supply to continue its growth beyond a few millimeters. Suppression of angiogenesis is therefore an important means for anti-cancer therapeutic intervention. On the other hand, inflammation is implicated in a board array of diseases and disorders, such as systemic lupus erythematosus (SLE), diabetes, chronic renal disease, asthma, autoimmune disease, chronic inflammation, chronic prostatitis, glomerulonephritis, hypersensitivities and allergies, skin disorders such as eczema, inflammatory bowel disease, pelvic inflammatory disease, reperfusion injury, rheumatoid arthritis, transplant rejection (e.g., graft versus host disease), cytokine storm syndrome, secondary hemophagocytic lymphohistiocytosis, sepsis, macrophage activation syndrome, and vasculitis. Because of the high prevalence of inflammation in human health conditions and the critical importance of angiogenesis in tumorigenesis, there exists an urgent need for the development of new and effective therapeutics targeting inflammation and reducing or eliminating undesirable effects of inappropriate angiogenesis. The present invention fulfills this and other related needs.

### BRIEF SUMMARY OF THE INVENTION

This invention provides new methods and compositions useful for inhibiting angiogenesis and inflammation based on the discovery that the interaction between the heparin-binding domain (HBD) of VEGF165 and integrin αvβ3, especially during the formation of ternary complex with VEGF receptor-2 (VEGFR-2 or KDR), plays an important role in integrin-mediated signaling leading to angiogenesis and inflammation. Thus, in one aspect, the present invention provides an isolated polypeptide comprising the amino acid sequence of SEQ ID NO:1 the 111-165 segment of which contains at least one modification or mutation, such that the polypeptide has a reduced or abolished ability to bind integrin αvβ3 compared with the binding between wild-type VEGF165 (amino acid sequence set forth in SEQ ID NO:1) and integrin αvβ3. The mutation may be substitution, deletion, or addition at one or more amino acid residues of the 111-165 segment of SEQ ID NO:1, including up to deletion of about 10, 15, 20, 25, 30, 35, 40, or 50 amino acids of the segment or the entire segment. In some embodiments, the mutation is a substitution, for example, a substitution of the original amino acid in SEQ ID NO:1 with an alanine (A) or with a glutamic acid (E). In some embodiments, the mutation or mutations are at one or multiple of residues 123, 124, 125, 140, 145, and 149, for example, only one mutation is at one residue 123, 124, or 125, e.g., substituted with an A or E at each position; or the mutations are at all three residues 123, 124, and 125, e.g., substituted with an A or E at each position; or the mutations are at residues 140, 145, and 149, e.g., substituted with an A or E at each position. In some embodiments, the modification to the amino acid sequence of SEQ ID NO:1 includes one or more substitutions at residue 123, 124, 125, 140, 145, or 149. In some embodiments, the substitution is an A substitution, replacing the original amino acid in SEQ ID NO: 1. In some embodiments, the substitution is an E substitution, replacing the original amino acid in SEQ ID NO: 1. In some embodiments, the polypeptide comprises an A or E substitution at each of residues 123, 124, 125, 140, 145, and 149. In some embodiments, the polypeptide comprises an A substitution at each of residues 123, 124, 125, 140, 145, and 149. In some embodiments, the polypeptide comprises SEQ ID NO:2 and a heterologous amino acid sequence. In some embodiments, the polypeptide consists of SEQ ID NO:2, optionally fused with one or more heterologous peptide sequence to form a longer fusion protein, for example, with one heterologous amino acid sequence located at either or both N-terminus and C-terminus. In some embodiments, the polypeptide is conjugated with a heterologous moiety such as a detectable moiety for easy detection, an affinity moiety for easy purification (e.g., 6×His or glutathione S-transferase, GST), or a solid support. In some embodiments, the polypeptide or its fusion protein further comprises modification such as substitution with one or more D-amino acid(s) or PEGlyation (covalent attachment or amalgamation of polyethylene glycol (PEG) polymer chains) at one or more residues, which may be directly PEGylated or substituted with another amino acid such as Lys, which permits PEGlyation. PEGylation can take place on amino acids including lysine, cysteine, histidine, arginine, aspartic acid, glutamic acid, serine, threonine, tyrosine. Further, the N-terminal amino group and the C-terminal carboxylic acid of the peptide or its fusion protein can also be used, directly or upon functionalization, as a site for PEGylation. In some embodiments, the polypeptide or its conjugate is a part of a composition further comprising one or more physiologically or pharmaceutically acceptable excipients.

In a second aspect, the present invention provides a nucleic acid comprising a polynucleotide sequence encoding the polypeptide of this invention or its related fusion protein as described above and herein. Also provided are an expression cassette comprising such a polynucleotide sequence operably linked to a promoter, for example, a heterologous promoter, which directs the expression of the polypeptide of this invention or its related fusion protein, a vector comprising the expression cassette, as well as a host cell comprising the expression cassette or the vector. In some embodiments, the nucleic acid, the expression cassette, the vector, or the host cell is present in a composition further comprising one or more physiological or pharmaceutically acceptable excipients.

In a third aspect, the present invention provides a method for treating cancer by suppressing cancer cell proliferation and/or metastatic potential or a method for treating an inflammatory condition. The method includes a step of administering to a subject in need thereof an effective amount of a composition comprising the polynucleotide of this invention as described above and herein or the nucleic acid comprising a coding sequence to express the polypeptide. In some embodiments, the composition is administered systemically (e.g., by injection or oral ingestion) or locally (e.g., by topical application or by suppository). In some embodiments, the composition is administered to the subject by intravenous, subcutaneous, intraperitoneal, intraosseous, intramuscular, or intratumoral injection. In some embodiments, the composition is administered orally or nasally or topically. In some embodiments, the subject is suffering from or at risk of developing a condition involving inappropriate angiogenesis, such as cancer, including metastatic cancer. In some embodiments, the subject is suffering from or at risk of developing an inflammatory condition. In some embodiments, one or more signs, symptoms, and/or sequelae associated with the condition to be treated are mitigated in the subject.

In a fourth aspect, the present invention provides a method for identifying a modulator (e.g., an inhibitor or enhancer of VEGF-integrin αvβ3 binding). Such modulator, especially inhibitor may be useful as a therapeutic agent for treating an inflammatory disease or a condition involving excessive/inappropriate angiogenesis such as cancer. The method comprising the steps of (1) contacting integrin αvβ3 and a polypeptide comprising the 111-165 segment of SEQ ID NO: 1, in the presence of a test compound, under conditions permissible for VEGF-integrin αvβ3 binding; and (2) detecting the level of polypeptide-integrin αvβ3 binding, wherein a decrease in the level of binding when compared with the level of binding in the absence of the test compound indicates the compound as an inhibitor of VEGF-integrin αvβ3 binding. Conversely, an increase in the binding level identifies the test compound as an enhancer of the VEGF-integrin αvβ3 binding. In some embodiments, the polypeptide consists of SEQ ID NO:1 or the 111-165 segment of SEQ ID NO:1. In some embodiments, the polypeptide comprises (1) SEQ ID NO:1 or the 111-165 segment of SEQ ID NO: 1; and (2) one or more heterologous amino acid sequence. In some embodiments, the heterologous amino acid sequence is an affinity tag, such as a GST or a string of 6-10 His. In some embodiments, the integrin αvβ3 is expressed on a cell surface, whereas in other embodiments, the steps (1) and (2) are performed with the polypeptide and integrin αvβ3 in a cell-free in vitro system, for example, either one of the integrin αvβ3 and the polypeptide is immobilized on a solid substrate.

In a fifth aspect, the present invention provides a kit for inhibiting inflammation or for inhibiting cancer progression by suppressing angiogenesis. The kit includes a first container containing the composition of this invention as described above or herein and a second container containing a second therapeutic agent (e.g., another anti-inflammatory therapeutic agent or another anti-cancer therapeutic agent). Optionally, the kit further includes user instruction material providing description of dosing arrangements and its intended use.

### BRIEF DESCRIPTION OF THE DRAWINGS

FIG. 1 Integrin αvβ3 binds to the heparin binding domain (HBD, residues 111-165) of VEGF165, but not to the N-terminal domain (NTD, residues 1-110). FIG. 1a-b: Interaction of soluble αvβ3 with immobilized VEGF165 HBD vs NTD. Microtiter wells were coated with increasing concentrations of VEGF165 or its fragments and incubated with soluble αvβ3, after blocking non-specific protein binding sites with BSA. In (b) control wells were coated with BSA. αvβ3 binding was detected with non-function blocking anti-β3 mAb AV10. FIG. 1c: Specificity of αvβ3 binding to the VEGF165-HBD documented based on inhibition of αvβ3 binding by function-blocking anti-β3 antibody, mAb 7E3, or heat treatment, but not by control mouse IgG (mIgG). Control wells were coated with BSA. FIG. 1d: Cation-dependence of αvβ3 binding to the VEGF165-HBD. Binding of soluble αvβ3 to HBD protein (10 g/ml) immobilized on microtiter plates and blocked with BSA, in the absence of divalent cations (EDTA) or presence of Mn²⁺, Ca²⁺, or Mg²⁺(1 mM). Control wells were coated with BSA. FIG. 1e: Surface Plasmon Resonance analysis of αvβ3-HBD interaction. αvβ3 protein was immobilized to a sensor chip and binding of solubilized mobile HBD protein was measured as analyte at increasing concentrations. FIG. 1f: Model of αvβ3 binding to the C-terminal HBD within a VEGF165 homodimer formed by two anti-parallel VEGF164 monomers known to interact via their N-terminal domains (NTD). The NTD does not contribute to αvβ3 binding as documented in our study. Where applicable, data are shown as means+/−SD of triplicate experiments.

FIG. 2 Mapping the integrin binding sites within the heparin-binding domain (HBD) of VEGF165. FIG. 2a: Docking model of the interaction between integrin αvβ3 and the HBD based on docking simulation using Autodock 3. HBD amino acid residues predicted to contribute to αvβ3 binding are Arg-123, Arg-124, Lys-125, Lys-140, Arg-145, and Arg-149. FIG. 2b: Position of HBD amino acids within the predicted integrin binding interface were selected for mutagenesis and changed to Ala in combinations. FIG. 2c: Binding of soluble αvβ3 to VEGF165-HBD mutants coated onto microtiter wells at increasing concentrations. Non-specific binding sites were blocked with BSA. FIG. 2d: Binding of soluble αvβ3 to VEGF165-HBD mutants, coated at near saturation concentration (2.5 g/ml) identified for the wild type (WT) HBD protein, revealed that all HBD amino acids predicted to contribute to αvβ3 binding are required for VEGF165-integrin αvβ3 interaction. Where applicable, data are shown as means+/−SD of triplicate experiments.

FIG. 3 Partial characterization of the full-length VEGF165 mutant defective in integrin binding. FIG. 3a: SDS-PAGE analysis of wild type and mutant VEGF165. In mutant VEGF165 all six amino acids within the HBD identified as required for integrin αvβ3 binding were changed to Ala (R123A/R124A/K125A/K140A/R145A/R149A). Molecular size values in kDa. FIG. 3b: Pull-down of VEGF165 by KDR. His-tagged KDR was immobilized on Ni-NTR beads and incubated with full-length VEGF165 wild type (WT) vs VEGF165 mutant (mut) protein in binding buffer for 2 h at 4 C, before elution and analysis of the retained protein by SDS-PAGE. Both wild type and mutant VEGF165 bind to KDR. FIG. 3c: ERK1/2 activation in HUVECs by VEGF165. Starved HUVECs in M200 basal medium without low serum growth supplement were treated with VEGF165 (10 ng/ml) for 10 min before lysis and Western blot analysis. Wild-type (WT) but not mutant (mut) VEGF165 activates ERK1/2 phosphorylation in human umbilical vein endothelial cells (HUVEC). FIG. 3d: Dose dependence of ERK1/2 activation in HUVECs by VEGF165. FIG. 3e: Integrin αvβ3 phosphorylation in HUVEC in response to VEGF165. Starved HUVECs were treated with VEGF165 wild type vs mutant (10 ng/ml) before lysis and Western blot analysis of β3 integrin subunit phosphorylation. Wild-type (WT) but not mutant (mut) VEGF165 activates integrin αvβ3 phosphorylation in HUVECs. FIG. 3f: KDRY1175 phosphorylation in HUVEC in response to VEGF165. In HUVECs, wild-type (WT) but not mutant (mut) VEGF165 activates KDR Y1175 phosphorylation known to stimulate endothelial cell proliferation and migration. Starved HUVECs were treated for Western blot analysis as in panel e.

FIG. 4 The effect of Glu mutations in the heparin-binding domain on integrin binding. Several basic amino acid residues on the surface of the heparin-binding domain were mutated to Glu. FIG. 4(a) Adhesion of αvβ3-K562 cells and K562 cells to the heparin-binding domain mutants were shown. FIG. 4(b) Positions of amino acid residues of HBD that are involved in HBD-integrin αvβ3 interaction. FIG. 4(c) Effects of HBD mutations on adhesion of αvβ3-K562 cells to HBD were shown. FIG. 4(d) Effects of HBD individual point mutations in amino acid residues at positions 123-125 on adhesion of αvβ3-K562 to HBD were shown. FIG. 4(e) Effects of HBD mutations on adhesion of K562 cells to HBD were shown. FIG. 4(f) Effects of HBD individual point mutations in amino acid residues at positions 123-125 on adhesion of αvβ3-K562 to HBD were shown. The wells of 96-well microtiter plate were coated with His-tagged the heparin-binding domain and were incubated with K562 cells or αvβ3-K562 cells. Bound cells were quantified using endogenous phosphatase activity. The individual R123E, R124E, and K125E mutants were as defective in αvβ3 binding as was the R123E/R124E/K125E mutant.

### DEFINITIONS

The terms “a,” “an,” and “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The terms “about” and “approximately” as used herein shall generally mean an acceptable degree of error for the quantity measured given the nature or precision of the measurements. These terms generally denotes a range of 10% of a given value. For example, “about 10” means a range of 10±1 (10×10%), i.e., 9-11.

As used herein, the terms “VEGF dominant negative polypeptide,” “VEGF dominant negative mutant,” “VEGF dominant negative mutant polypeptide,” and “VEGF mutant polypeptide” refer to a VEGF165 antagonist compound in the form of a mutated VEGF165 polypeptide, or a fragment thereof, which suppresses VEGF165/integrin-induced cellular signaling by way of its interaction with integrins (such as integrin αvβ3) in a manner that imposes an inhibitory or disruptive effect on the specific binding among wild-type VEGF165 and integrins, thus inhibiting downstream events normally triggered by VEGF165-integrin interaction and subsequent signaling, for example, VEGF165-mediated angiogenesis or inflammatory response. In an exemplary VEGF165 dominant negative mutant, one or more amino acid residues predicted to interact with integrin, e.g., at least one possibly multiple residues within the heparin-binding domain (HBD, 111-165 segment of SEQ ID NO: 1), are mutated, for example, by deletion or by substitution with a different amino acid (e.g., mutations at one or more residues 123, 124, 125, 140, 145, and 149 of SEQ ID NO: 1), resulting in the mutant having decreased or even abolished capability to bind integrin such as αvβ3. These VEGF dominant negative mutants can be identified based on their deficiency compared to the wild-type VEGF in decreased integrin (e.g., integrin αvβ3) binding by at least about 10%, 20%, 30%, 40%, 50%, 60%, 70%, 80%, or 90% as compared to the wild-type VEGF165 protein (SEQ ID NO: 1).

A VEGF dominant negative mutant may be initially generated based on a wild-type VEGF165 amino acid sequence (e.g., SEQ ID NO: 1) with certain amino acid residue(s) (e.g., residues 123, 124, 125, 140, 145, and 149) mutated. In some embodiments, the VEGF165 dominant negative mutant comprises an amino acid sequence having at least about 80% (e.g., at least about 80%, 81%, 82%, 83%, 84%, 85%, 86%, 87%, 88%, 89%, 90%, 91%, 92%, 93%, 94%, 95%, 96%, 97%, 98%, or 99%) identity to SEQ ID NO:1 in which at least one, maybe two, three, four, five, or six, of residues 123, 124, 125, 140, 145, and 149 are mutated, for example, replacing the original amino acids with an alanine. In some embodiments, the VEGF dominant negative mutant polypeptide comprises or consists of the amino acid sequence set forth in SEQ ID NO:2. In some embodiments, the VEGF dominant negative mutant polypeptide consists of the amino acid sequence set forth in SEQ ID NO:1 in which at least about 5, 10, 15, 20, 25, 30, 35, 40, 45, or 50 residues of the 111-165 segments are deleted or the entire segment is deleted.

Furthermore, the VEGF dominant negative mutant polypeptide may further include one or more heterologous amino acid sequences (derived from a source other than the VEGF protein) at its N-terminus and/or C-terminus. For example, a VEGF165 dominant negative mutant may optionally include one or more additional heterologous amino acid sequence(s) of 1, 2, 3, 4, 5, 6, 7, 8, 9, 10, 11, 12, 13, 14, 15, 16, 17, 18, 19, 20, 25, 30, 35, 40, 45, or up to 50 amino acids at the C- and/or N-terminus of the VEGF-derived sequence. In some embodiments, the one or more heterologous amino acid(s) comprise a cysteine residue that is located at the N- and/or C-terminal end and may be used, for example, to attach PEG group(s). Such heterologous peptide sequences can be of a varying nature, for example, any one of the “tags” known and used in the field of recombinant proteins: a peptide tag such as an AviTag, a peptide allowing biotinylation by the enzyme BirA and so the protein can be isolated by streptavidin, a Calmodulin-tag, a peptide bound by the protein calmodulin, a polyglutamate tag, a peptide binding efficiently to anion-exchange resin such as Mono-Q, an E-tag, a peptide recognized by an antibody, a FLAG-tag, a peptide recognized by an antibody, an HA-tag, a peptide recognized by an antibody, a His-tag, 5-10 histidines bound by a nickel or cobalt chelate, a Myc-tag, a short peptide recognized by an antibody, an S-tag, an SBP-tag, a peptide that specifically binds to streptavidin, a Softag 1 for mammalian expression, a Softag 3 for prokaryotic expression, a Strep-tag, a peptide that binds to streptavidin or the modified streptavidin called streptactin (Strep-tag II), a TC tag, a tetracysteine tag that is recognized by FlAsH and ReAsH biarsenical compounds, a V5 tag, a peptide recognized by an antibody, a VSV-tag, a peptide recognized by an antibody, an Xpress tag; or a covalent peptide tags such as an Isopeptag, a peptide that binds covalently to pilin-C protein, a SpyTag, a peptide that binds covalently to SpyCatcher protein; or a protein tag such as a BCCP tag (Biotin Carboxyl Carrier Protein), a protein domain biotinylated by BirA enabling recognition by streptavidin, a Glutathione-S-transferase (GST) tag, a protein that binds to immobilized glutathione, a Green fluorescent protein (GFP) tag, a protein that is spontaneously fluorescent and can be bound by nanobodies, a Maltose binding protein (MBP) tag, a protein that binds to amylose agarose, a Nus-tag, a Thioredoxin-tag, an Fc-tag (derived from immunoglobulin Fc domain, allowing dimerization and solubilization), a tag that can be used for purification on Protein-A Sepharose; as well as other types of tags such as the Ty tag. Furthermore, the CD40L dominant negative mutants may also include one or more D-amino acids or include chemical modifications such as PEGylation, myristoylation, glycosylation, crosslinking, and the like.

In some embodiments, the VEGF dominant negative mutant polypeptide is present as part of a fusion protein, e.g., a fusion protein comprising a VEGF dominant negative mutant polypeptide described herein and an Fc polypeptide. As used herein, the term “Fc polypeptide” refers to the C-terminal region of an immunoglobulin heavy chain polypeptide. An Fc polypeptide typically contains constant region sequences (e.g., the CH2 domain and/or the CH₃ domain) and may also contain the hinge region (or a portion thereof). An Fc polypeptide typically does not contain a variable region. In some embodiments, the Fc polypeptide is an IgG1, IgG2, IgG3, or IgG4 Fc polypeptide.

Furthermore, the fusion protein may be labeled, e.g., with a radionuclide. The term “radionuclide” is intended to include any nuclide that exhibits radioactivity. A “nuclide” refers to a type of atom specified by its atomic number, atomic mass, and energy state, such as carbon 14 (¹⁴C). “Radioactivity” refers to the radiation, including alpha particles, beta particles, nucleons, electrons, positrons, neutrinos, and gamma rays, emitted by a radioactive substance. Examples of radionuclides suitable for use in the present invention include, but are not limited to, fluorine 18 (¹⁸F), phosphorus 32 (³²P), scandium 47 (⁴⁷Sc), cobalt 55 (⁵⁵Co), copper 60 (⁶⁰Cu), copper 61 (⁶¹Cu), copper 62 (⁶²Cu), copper 64 (⁶⁴Cu), gallium 66 (⁶⁶Ga), copper 67 (⁶⁷Cu), gallium 67 (⁶⁷Ga), gallium 68 (⁶⁸Ga), rubidium 82 (⁸²Rb), yttrium 86 (⁸⁶Y), yttrium 87 (⁸⁷Y), strontium 89 (⁸⁹Sr), yttrium 90 (⁹⁰Y), rhodium 105 (¹⁰⁵Rh) silver 111 (¹¹¹Ag), indium 111 (¹¹¹In), iodine 124 (1241), iodine 125 (1251), iodine 131 (¹³¹I), tin 117m (^{17m}Sn), technetium 99m (^{99m}Tc), promethium 149 (¹⁴⁹Pm), samarium 153 (¹⁵³Sm), holmium 166 (¹⁶⁶Ho), lutetium 177 (¹⁷⁷Lu), rhenium 186 (¹⁸⁶Re), rhenium 188 (¹⁸⁸Re), thallium 201 (²⁰¹Tl), astatine 211 (²¹¹At), and bismuth 212 (²¹²Bi). As used herein, the “in” in ^{117m}Sn and ^{99m}Tc stands for the meta state. Additionally, naturally-occurring radioactive elements such as uranium, radium, and thorium, which typically represent mixtures of radioisotopes, are suitable examples of radionuclides. ⁶⁷Cu, ¹³¹I, ¹⁷⁷Lu, and ¹⁸⁶Re are beta- and gamma-emitting radionuclides. ²¹²Bi is an alpha- and beta-emitting radionuclide. ²¹¹At is an alpha-emitting radionuclide. ³²P, ⁴⁷Sc, ⁸⁹Sr, ⁹⁰Y, ¹⁰⁵Rh, ¹¹¹Ag, ^{117m}Sn, ¹⁴⁹Pm, ¹⁵³Sm, ¹⁶⁶Ho, and ¹⁸⁸Re are examples of beta-emitting radionuclides. ⁶⁷Ga, ¹¹¹In ^{99m}Tc, and ²⁰¹Tl are examples of gamma-emitting radionuclides. ⁵⁵Co, ⁶⁰Cu, ⁶¹Cu, ⁶²Cu, ⁶⁶Ga, ⁶⁸Ga, ⁸²Rb, and ⁸⁶Y are examples of positron-emitting radionuclides. ⁶⁴Cu is a beta- and positron-emitting radionuclide.

In some embodiments, a modification such as PEGylation or myristoylation, or fusion to an Fc polypeptide, increases the half-life (e.g., in the body of a subject such as a mammal) of the polypeptide, as compared to a corresponding VEGF dominant negative mutant polypeptide that does not have the modification of that is not fused to the Fc polypeptide. Increased half-life can be due to, for example, increased stability (i.e., the polypeptide is more resistant to degradation and/or metabolism) and/or decreased clearance (e.g., renal clearance). In some embodiments, half-life of the modified (e.g., PEGylated and/or myristoylated) polypeptide is increased by at least about 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 15-fold, 20-fold, 25-fold, 30-fold, 35-fold, 40-fold, 45-fold, 50-fold, 60-fold, 70-fold, 80-fold, 90-fold, 100-fold, or more.

The term “inhibiting” or “inhibition,” as used herein, refers to any detectable negative effect on a target biological process, such as the binding between VEGF165 and integrin αvβ3, or on its downstream processes including inflammatory response or angiogenesis. Typically, an inhibition is reflected in a decrease of at least 10%, 20%, 30%, 40%, 50%, 60%, 70%, 80% or higher in VEGF165-integrin binding, or any one of the downstream parameters mentioned above, when compared to a control. The terms “reducing,” “reduction,” “decreasing,” and “decrease” are used in a similar fashion.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” means the segment of DNA involved in producing a polypeptide chain. It may include regions preceding and following the coding region (leader and trailer) as well as intervening sequences (introns) between individual coding segments (exons).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, whereas non-naturally occurring amino acids include D-amino acids and those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds having a structure that is different from the general chemical structure of an amino acid, but that functions in a manner similar to a naturally occurring amino acid.

There are various known methods in the art that permit the incorporation of an unnatural amino acid derivative or analog into a polypeptide chain in a site-specific manner, see, e.g., WO 02/086075.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

In the present application, amino acid residues are numbered according to their relative positions from the left most residue, which is numbered 1, in an unmodified wild-type polypeptide sequence.

As used in herein, the terms “identical” or percent “identity,” in the context of describing two or more polynucleotide or amino acid sequences, refer to two or more sequences or subsequences that are the same or have a specified percentage of amino acid residues or nucleotides that are the same (for example, a mutant VEGF amino acid sequence has at least 80% identity, preferably 85%, 90%, 91%, 92%, 93, 94%, 95%, 96%, 97%, 98%, 99%, or 100% identity, to a reference sequence, e.g., a wild-type VEGF protein having the amino acid sequence set forth in SEQ ID NO: 1), when compared and aligned for maximum correspondence over a comparison window, or designated region as measured using one of the following sequence comparison algorithms or by manual alignment and visual inspection. Such sequences are then said to be “substantially identical.” With regard to polynucleotide sequences, this definition also refers to the complement of a test sequence. Preferably, the identity exists over a region that is at least about 50 amino acids or nucleotides in length, or more preferably over a region that is 75-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.0 algorithms and the default parameters discussed below are used.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Current Protocols in Molecular Biology (Ausubel et al., eds. 1995 supplement)).

Examples of algorithms that are suitable for determining percent sequence identity and sequence similarity are the BLAST and BLAST 2.0 algorithms, which are described in Altschul et al., (1990) J. Mol. Biol. 215: 403-410 and Altschul et al. (1977) Nucleic Acids Res. 25: 3389-3402, respectively. Software for performing BLAST analyses is publicly available at the National Center for Biotechnology Information website, ncbi.nlm.nih.gov. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin and Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

“Polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

The term “effective amount,” as used herein, refers to an amount that produces therapeutic effects for which a substance is administered. The effects include the prevention, correction, or inhibition of progression of the symptoms of a disease/condition and related complications to any detectable extent. The exact amount will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); and Pickar, Dosage Calculations (1999)).

An “expression cassette” is a nucleic acid construct, generated recombinantly or synthetically, with a series of specified nucleic acid elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter.

The term “inflammation” refers to an organism's (e.g., a mammal's) immune response to irritation, toxic substances, pathogens, or other stimuli. The response can involve innate immune components and/or adaptive immunity. Inflammation is generally characterized as either chronic or acute. Acute inflammation can be characterized by, as non-limiting examples, redness, pain, heat, swelling, and/or loss of function due to infiltration of plasma proteins and leukocytes to the affected area. Chronic inflammation can be characterized by, as non-limiting examples, persistent inflammation, tissue destruction, and/or attempts at repair. Monocytes, macrophages, plasma B cells, and other lymphocytes are commonly recruited to the affected area, and angiogenesis and fibrosis can occur, in some instances leading to scar tissue.

The term “inflammatory condition” or “inflammatory disorder” refers to a condition or disorder that is characterized or exacerbated by an inflammatory response, as described above. A list of exemplary inflammatory conditions includes: systemic lupus erythematosus (SLE), diabetes, chronic renal disease, asthma, autoimmune disease, chronic inflammation, chronic prostatitis, glomerulonephritis, hypersensitivities and allergies, skin disorders such as eczema, inflammatory bowel disease, pelvic inflammatory disease, reperfusion injury, rheumatoid arthritis, transplant rejection, and vasculitis.

The term “cancer” refers to any of various malignant neoplasms characterized by the proliferation of anaplastic cells that tend to invade surrounding tissue and metastasize to new body sites. Non-limiting examples of different types of cancer suitable for treatment using the compositions and methods of the present invention include colorectal cancer, colon cancer, anal cancer, liver cancer, ovarian cancer, breast cancer, lung cancer, bladder cancer, thyroid cancer, pleural cancer, pancreatic cancer, cervical cancer, prostate cancer, testicular cancer, bile duct cancer, gastrointestinal carcinoid tumors, esophageal cancer, gall bladder cancer, rectal cancer, appendix cancer, small intestine cancer, stomach (gastric) cancer, renal cancer (e.g., renal cell carcinoma), cancer of the central nervous system, skin cancer, oral squamous cell carcinoma, choriocarcinomas, head and neck cancers, bone cancer, osteogenic sarcomas, fibrosarcoma, neuroblastoma, glioma, melanoma, leukemia (e.g., acute lymphocytic leukemia, chronic lymphocytic leukemia, acute myelogenous leukemia, chronic myelogenous leukemia, or hairy cell leukemia), lymphoma (e.g., non-Hodgkin's lymphoma, Hodgkin's lymphoma, B-cell lymphoma, or Burkitt's lymphoma), and multiple myeloma.

The term “heterologous,” as used in the context of describing the relative location or position of two elements, such as two polynucleotide sequences (e.g., a promoter and a polypeptide-encoding sequence) or polypeptide sequences (e.g., a first amino acid sequence (such as one set forth in SEQ ID NO:1 with a mutation or mutations) and a second peptide sequence serving as a fusion partner with the first amino acid sequence), means that the two elements are not naturally found in the same relative location or position. Thus, a “heterologous promoter” of a gene refers to a promoter that is not naturally operably linked to that gene. Similarly, a “heterologous polypeptide/amino acid sequence” or “heterologous polynucleotide” to a VEGF165 amino acid sequence or its encoding sequence is one derived from anon-VEGF165 origin.

A composition “consisting essentially of a VEGF dominant negative mutant” is one that includes a VEGF165 mutant that inhibits specific binding between wild-type VEGF165 and integrin (such as integrin αvβ3) but no other compounds that contribute significantly to the inhibition of such binding. Such compositions may include one or more inactive physiologically or pharmaceutically acceptable excipients or carriers, e.g., for formulation or stability of a pharmaceutical composition, or active ingredients that do not significantly contribute to the inhibition of VEGF165-integrin binding. Exemplary compositions consisting essentially of a VEGF dominant negative mutant (e.g., a polypeptide R123A/R124A/K125A/K140A/R145A/R149A of SEQ ID NO: 1, R123A/R124A/K125A of SEQ ID NO: 1, or K140A/R145A/R149A of SEQ ID NO: 1) include therapeutics, medicaments, and pharmaceutical compositions.

The term “subject” or “subject in need of treatment” refers to an individual who seeks medical attention due to risk of, or actual sufferance from, a condition involving undesirable inflammation or a condition involving undesirable angiogenesis (e.g., cancer). The term subject can include both animals, especially mammals, and humans. Subjects or individuals in need of treatment include those that demonstrate symptoms of an inflammatory disorder or cancer or those are at risk of later developing the disease or disorder and/or its symptoms.

### DETAILED DESCRIPTION OF THE INVENTION

### I. Introduction

Efforts have been made to develop dominant-negative VEGF165 mutants using a strategy that was successfully used to develop mutants of other proteins that normally participate in an integrin-containing signaling complex. The mutants have diminished ability to bind integrin while retaining the ability to bind other members of the signaling complex and therefore act as dominant-negative mutants to the wild-type protein in suppressing signaling mediated by the wild-type protein and integrin. The potential of such dominant-negative mutants as therapeutic agents against the downstream events of cellular signaling mediated by the wild-type protein, such as inflammation and angiogenesis, is explored.

### II. Production of Mutant VEGF165 Polypeptides

### A. General Recombinant Technology

Basic texts disclosing general methods and techniques in the field of recombinant genetics include Sambrook and Russell, Molecular Cloning, A Laboratory Manual (3rd ed. 2001); Kriegler, Gene Transfer and Expression: A Laboratory Manual (1990); and Ausubel et al., eds., Current Protocols in Molecular Biology (1994).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage & Caruthers, Tetrahedron Lett. 22: 1859-1862 (1981), using an automated synthesizer, as described in Van Devanter et. al., Nucleic Acids Res. 12: 6159-6168 (1984). Purification of oligonucleotides is performed using any art-recognized strategy, e.g., native acrylamide gel electrophoresis or anion-exchange HPLC as described in Pearson & Reanier, J. Chrom. 255: 137-149 (1983).

The sequence of a VEGF165 gene, a polynucleotide encoding a polypeptide having the amino acid sequence SEQ ID NO:1, and synthetic oligonucleotides can be verified after cloning or subcloning using, e.g., the chain termination method for sequencing double-stranded templates of Wallace et al., Gene 16: 21-26 (1981).

### B. Coding Sequence for a VEGF Mutant Polypeptide

Polynucleotide sequences encoding a wild-type VEGF165 protein, especially a wild-type human VEGF165 protein, have been determined and may be obtained from a commercial supplier. For example, the GenBank Accession Nos. for human VEGF165 mRNA and protein sequences are NM_001171626 and NP_001165097, respectively.

The rapid progress in the studies of human genome has made possible a cloning approach where a human DNA sequence database can be searched for any gene segment that has a certain percentage of sequence homology to a known nucleotide sequence, such as one encoding a previously identified human VEGF165 protein. Any DNA sequence so identified can be subsequently obtained by chemical synthesis and/or a polymerase chain reaction (PCR) technique such as overlap extension method. For a short sequence, completely de novo synthesis may be sufficient; whereas further isolation of full length coding sequence from a human cDNA or genomic library using a synthetic probe may be necessary to obtain a larger gene.

Alternatively, a nucleic acid sequence encoding a human VEGF165 protein can be isolated from a human cDNA or genomic DNA library using standard cloning techniques such as polymerase chain reaction (PCR), where homology-based primers can often be derived from a known nucleic acid sequence encoding a VEGF165. Most commonly used techniques for this purpose are described in standard texts, e.g., Sambrook and Russell, supra.

cDNA libraries suitable for obtaining a coding sequence for a human VEGF165 may be commercially available or can be constructed. The general methods of isolating mRNA, making cDNA by reverse transcription, ligating cDNA into a recombinant vector, transfecting into a recombinant host for propagation, screening, and cloning are well known (see, e.g., Gubler and Hoffman, Gene, 25: 263-269 (1983); Ausubel et al., supra). Upon obtaining an amplified segment of nucleotide sequence by PCR, the segment can be further used as a probe to isolate the full length polynucleotide sequence encoding the VEGF165 from the cDNA library. A general description of appropriate procedures can be found in Sambrook and Russell, supra.

A similar procedure can be followed to obtain a full-length sequence encoding a human VEGF165 from a human genomic library. Human genomic libraries are commercially available or can be constructed according to various art-recognized methods. In general, to construct a genomic library, the DNA is first extracted from a tissue where a VEGF165 is likely found. The DNA is then either mechanically sheared or enzymatically digested to yield fragments of about 12-20 kb in length. The fragments are subsequently separated by gradient centrifugation from polynucleotide fragments of undesired sizes and are inserted in bacteriophage λ vectors. These vectors and phages are packaged in vitro. Recombinant phages are analyzed by plaque hybridization as described in Benton and Davis, Science, 196: 180-182 (1977). Colony hybridization is carried out as described by Grunstein et al., Proc. Natl. Acad. Sci. USA, 72: 3961-3965 (1975).

Based on sequence homology, degenerate oligonucleotides can be designed as primer sets and PCR can be performed under suitable conditions (see, e.g., White et al., PCR Protocols: Current Methods and Applications, 1993; Griffin and Griffin, PCR Technology, CRC Press Inc. 1994) to amplify a segment of nucleotide sequence from a cDNA or genomic library. Using the amplified segment as a probe, the full-length nucleic acid encoding a VEGF165 is obtained.

Upon acquiring a nucleic acid sequence encoding a VEGF165 protein, the coding sequence can be further modified by a number of well-known techniques such as restriction endonuclease digestion, PCR, and PCR-related methods to generate coding sequences for VEGF mutants (especially the dominant-negative type). The polynucleotide sequence encoding a desired VEGF mutant polypeptide can then be subcloned into a vector, for instance, an expression vector, so that a recombinant polypeptide can be produced from the resulting construct. Further modifications to the coding sequence, e.g., nucleotide substitutions, may be subsequently made to alter the characteristics of the polypeptide.

A variety of mutation-generating protocols are established and described in the art, and can be readily used to modify a polynucleotide sequence encoding a VEGF165-related polypeptide. See, e.g., Zhang et al., Proc. Natl. Acad. Sci. USA, 94: 4504-4509 (1997); and Stemmer, Nature, 370: 389-391 (1994). The procedures can be used separately or in combination to produce variants of a set of nucleic acids, and hence variants of encoded polypeptides. Kits for mutagenesis, library construction, and other diversity-generating methods are commercially available.

Mutational methods of generating diversity include, for example, site-directed mutagenesis (Botstein and Shortle, Science, 229: 1193-1201 (1985)), mutagenesis using uracil-containing templates (Kunkel, Proc. Natl. Acad. Sci. USA, 82: 488-492 (1985)), oligonucleotide-directed mutagenesis (Zoller and Smith, Nucl. Acids Res., 10: 6487-6500 (1982)), phosphorothioate-modified DNA mutagenesis (Taylor et al., Nucl. Acids Res., 13: 8749-8764 and 8765-8787 (1985)), and mutagenesis using gapped duplex DNA (Kramer et al., Nucl. Acids Res., 12: 9441-9456 (1984)).

Other possible methods for generating mutations include point mismatch repair (Kramer et al., Cell, 38: 879-887 (1984)), mutagenesis using repair-deficient host strains (Carter et al., Nucl. Acids Res., 13: 4431-4443 (1985)), deletion mutagenesis (Eghtedarzadeh and Henikoff, Nucl. Acids Res., 14: 5115 (1986)), restriction-selection and restriction-purification (Wells et al., Phil. Trans. R. Soc. Lond. A, 317: 415-423 (1986)), mutagenesis by total gene synthesis (Nambiar et al., Science, 223: 1299-1301 (1984)), double-strand break repair (Mandecki, Proc. Natl. Acad. Sci. USA, 83: 7177-7181 (1986)), mutagenesis by polynucleotide chain termination methods (U.S. Pat. No. 5,965,408), and error-prone PCR (Leung et al., Biotechniques, 1: 11-15 (1989)).

### C. Modification of Nucleic Acids for Preferred Codon Usage in a Host Organism

The polynucleotide sequence encoding a VEGF mutant polypeptide can be further altered to coincide with the preferred codon usage of a particular host. For example, the preferred codon usage of one strain of bacterial cells can be used to derive a polynucleotide that encodes a recombinant polypeptide of the invention and includes the codons favored by this strain. The frequency of preferred codon usage exhibited by a host cell can be calculated by averaging frequency of preferred codon usage in a large number of genes expressed by the host cell (e.g., calculation service is available from web site of the Kazusa DNA Research Institute, Japan). This analysis is preferably limited to genes that are highly expressed by the host cell.

At the completion of modification, the coding sequences are verified by sequencing and are then subcloned into an appropriate expression vector for recombinant production of the VEGF mutant polypeptides.

### D. Chemical Synthesis of VEGF Mutant Polypeptides

The amino acid sequence of human VEGF165 protein is provided (SEQ ID NO:1). A VEGF mutant polypeptide comprising one or more point mutants in the wild-type VEGF amino acid sequence (SEQ ID NO:1) thus can also be chemically synthesized using conventional peptide synthesis or other protocols well known in the art.

Polypeptides may be synthesized by solid-phase peptide synthesis methods using procedures similar to those described by Merrifield et al., J. Am. Chem. Soc., 85:2149-2156 (1963); Barany and Merrifield, Solid-Phase Peptide Synthesis, in The Peptides: Analysis, Synthesis, Biology Gross and Meienhofer (eds.), Academic Press, N.Y., vol. 2, pp. 3-284 (1980); and Stewart et al., Solid Phase Peptide Synthesis 2nd ed., Pierce Chem. Co., Rockford, Ill. (1984). During synthesis, N-α-protected amino acids having protected side chains are added stepwise to a growing polypeptide chain linked by its C-terminal and to a solid support, i.e., polystyrene beads. The peptides are synthesized by linking an amino group of an N-α-deprotected amino acid to an α-carboxy group of an N-α-protected amino acid that has been activated by reacting it with a reagent such as dicyclohexylcarbodiimide. The attachment of a free amino group to the activated carboxyl leads to peptide bond formation. The most commonly used N-α-protecting groups include Boc, which is acid labile, and Fmoc, which is base labile.

Materials suitable for use as the solid support are well known to those of skill in the art and include, but are not limited to, the following: halomethyl resins, such as chloromethyl resin or bromomethyl resin; hydroxymethyl resins; phenol resins, such as 4-(α-[2,4-dimethoxyphenyl]-Fmoc-aminomethyl)phenoxy resin; tert-alkyloxycarbonyl-hydrazidated resins, and the like. Such resins are commercially available and their methods of preparation are known by those of ordinary skill in the art.

Briefly, the C-terminal N-α-protected amino acid is first attached to the solid support. The N-α-protecting group is then removed. The deprotected α-amino group is coupled to the activated α-carboxylate group of the next N-α-protected amino acid. The process is repeated until the desired peptide is synthesized. The resulting peptides are then cleaved from the insoluble polymer support and the amino acid side chains deprotected. Longer peptides can be derived by condensation of protected peptide fragments. Details of appropriate chemistries, resins, protecting groups, protected amino acids and reagents are well known in the art and so are not discussed in detail herein (See, Atherton et al., Solid Phase Peptide Synthesis: A Practical Approach, IRL Press (1989), and Bodanszky, Peptide Chemistry, A Practical Textbook, 2nd Ed., Springer-Verlag (1993)).

### III. Expression and Purification of VEGF Mutant Polypeptides

Following verification of the coding sequence, a VEGF mutant polypeptide of the present invention can be produced using routine techniques in the field of recombinant genetics, relying on the polynucleotide sequences encoding the polypeptide disclosed herein.

### A. Expression Systems

To obtain high level expression of a nucleic acid encoding a VEGF165 mutant polypeptide of the present invention, one typically subclones a polynucleotide encoding the polypeptide into an expression vector that contains a strong promoter to direct transcription, a transcription/translation terminator and a ribosome binding site for translational initiation. Suitable bacterial promoters are well known in the art and described, e.g., in Sambrook and Russell, supra, and Ausubel et al., supra. Bacterial expression systems for expressing the polypeptide are available in, e.g., E. coli, Bacillus sp., Salmonella, and Caulobacter. Kits for such expression systems are commercially available. Eukaryotic expression systems for mammalian cells, yeast, and insect cells are well known in the art and are also commercially available. In one embodiment, the eukaryotic expression vector is an adenoviral vector, an adeno-associated vector, or a retroviral vector.

The promoter used to direct expression of a heterologous nucleic acid depends on the particular application. The promoter is optionally positioned about the same distance from the heterologous transcription start site as it is from the transcription start site in its natural setting. As is known in the art, however, some variation in this distance can be accommodated without loss of promoter function.

In addition to the promoter, the expression vector typically includes a transcription unit or expression cassette that contains all the additional elements required for the expression of the VEGF mutant polypeptide in host cells. A typical expression cassette thus contains a promoter operably linked to the nucleic acid sequence encoding the VEGF mutant polypeptide and signals required for efficient polyadenylation of the transcript, ribosome binding sites, and translation termination. The nucleic acid sequence encoding the VEGF mutant polypeptide is typically linked to a cleavable signal peptide sequence to promote secretion of the polypeptide by the transformed cell. Such signal peptides include, among others, the signal peptides from tissue plasminogen activator, insulin, and neuron growth factor, and juvenile hormone esterase of Heliothis virescens. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as GST and LacZ. Epitope tags can also be added to recombinant proteins to provide convenient methods of isolation, e.g., c-myc.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the SV40 early promoter, SV40 later promoter, metallothionein promoter, murine mammary tumor virus promoter, Rous sarcoma virus promoter, polyhedrin promoter, or other promoters shown effective for expression in eukaryotic cells.

Some expression systems have markers that provide gene amplification such as thymidine kinase, hygromycin B phosphotransferase, and dihydrofolate reductase. Alternatively, high yield expression systems not involving gene amplification are also suitable, such as a baculovirus vector in insect cells, with a polynucleotide sequence encoding the VEGF mutant polypeptide under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are optionally chosen such that they do not interfere with the replication of the DNA in eukaryotic cells, if necessary. Similar to antibiotic resistance selection markers, metabolic selection markers based on known metabolic pathways may also be used as a means for selecting transformed host cells.

When periplasmic expression of a recombinant protein (e.g., a VEGF165 mutant polypeptide of the present invention) is desired, the expression vector further comprises a sequence encoding a secretion signal, such as the E. coli OppA (Periplasmic Oligopeptide Binding Protein) secretion signal or a modified version thereof, which is directly connected to 5′ of the coding sequence of the protein to be expressed. This signal sequence directs the recombinant protein produced in cytoplasm through the cell membrane into the periplasmic space. The expression vector may further comprise a coding sequence for signal peptidase 1, which is capable of enzymatically cleaving the signal sequence when the recombinant protein is entering the periplasmic space. More detailed description for periplasmic production of a recombinant protein can be found in, e.g., Gray et al., Gene 39: 247-254 (1985), U.S. Pat. Nos. 6,160,089 and 6,436,674.

A person skilled in the art will recognize that various conservative substitutions can be made to any wild-type VEGF165 polypeptide, to produce a VEGF mutant polypeptide that, while still retaining the ability to bind KDR, does not trigger VEGF165-integrin downstream signaling. Moreover, modifications of a polynucleotide coding sequence may also be made to accommodate preferred codon usage in a particular expression host without altering the resulting amino acid sequence.

### B. Transfection Methods

Standard transfection methods are used to produce bacterial, mammalian, yeast, insect, or plant cell lines that express large quantities of a VEGF mutant polypeptide, which are then purified using standard techniques (see, e.g., Colley et al., J Biol. Chem. 264: 17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132: 349-351 (1977); Clark-Curtiss & Curtiss, Methods in Enzymology 101: 347-362 (Wu et al., eds, 1983).

Any of the well-known procedures for introducing foreign nucleotide sequences into host cells may be used. These include the use of calcium phosphate transfection, polybrene, protoplast fusion, electroporation, liposomes, microinjection, plasmid vectors, viral vectors and any of the other well-known methods for introducing cloned genomic DNA, cDNA, synthetic DNA, or other foreign genetic material into a host cell (see, e.g., Sambrook and Russell, supra). It is only necessary that the particular genetic engineering procedure used be capable of successfully introducing at least one gene into the host cell capable of expressing the VEGF mutant polypeptide.

### C. Purification of Recombinantly Produced VEGF Mutant Polypeptides

Once the expression of a recombinant VEGF mutant polypeptide in transfected host cells is confirmed, e.g., via an immunoassay such as Western blotting assay, the host cells are then cultured in an appropriate scale for the purpose of purifying the recombinant polypeptide.

1. Purification of Recombinantly Produced Polypeptides from Bacteria

When the VEGF mutant polypeptides of the present invention are produced recombinantly by transformed bacteria in large amounts, typically after promoter induction, although expression can be constitutive, the polypeptides may form insoluble aggregates. There are several protocols that are suitable for purification of protein inclusion bodies. For example, purification of aggregate proteins (hereinafter referred to as inclusion bodies) typically involves the extraction, separation and/or purification of inclusion bodies by disruption of bacterial cells, e.g., by incubation in a buffer of about 100-150 μg/ml lysozyme and 0.10% Nonidet P40, a non-ionic detergent. The cell suspension can be ground using a Polytron grinder (Brinkman Instruments, Westbury, NY). Alternatively, the cells can be sonicated on ice. Additional methods of lysing bacteria are described in Ausubel et al. and Sambrook and Russell, both supra, and will be apparent to those of skill in the art.

The cell suspension is generally centrifuged and the pellet containing the inclusion bodies resuspended in buffer which does not dissolve but washes the inclusion bodies, e.g., 20 mM Tris-HCl (pH 7.2), 1 mM EDTA, 150 mM NaCl and 2% Triton-X 100, a non-ionic detergent. It may be necessary to repeat the wash step to remove as much cellular debris as possible. The remaining pellet of inclusion bodies may be resuspended in an appropriate buffer (e.g., 20 mM sodium phosphate, pH 6.8, 150 mM NaCl). Other appropriate buffers will be apparent to those of skill in the art.

Following the washing step, the inclusion bodies are solubilized by the addition of a solvent that is both a strong hydrogen acceptor and a strong hydrogen donor (or a combination of solvents each having one of these properties). The proteins that formed the inclusion bodies may then be renatured by dilution or dialysis with a compatible buffer. Suitable solvents include, but are not limited to, urea (from about 4 M to about 8 M), formamide (at least about 80%, volume/volume basis), and guanidine hydrochloride (from about 4 M to about 8 M). Some solvents that are capable of solubilizing aggregate-forming proteins, such as SDS (sodium dodecyl sulfate) and 70% formic acid, may be inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of the immunologically and/or biologically active protein of interest. After solubilization, the protein can be separated from other bacterial proteins by standard separation techniques. For further description of purifying recombinant polypeptides from bacterial inclusion body, see, e.g., Patra et al., Protein Expression and Purification 18: 182-190 (2000).

Alternatively, it is possible to purify recombinant polypeptides, e.g., a VEGF165 mutant polypeptide, from bacterial periplasm. Where the recombinant protein is exported into the periplasm of the bacteria, the periplasmic fraction of the bacteria can be isolated by cold osmotic shock in addition to other methods known to those of skill in the art (see e.g., Ausubel et al., supra). To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

### 2. Standard Protein Separation Techniques for Purification

When a recombinant polypeptide of the present invention, e.g., a VEGF mutant polypeptide, is expressed in host cells in a soluble form, its purification can follow the standard protein purification procedure described below. This standard purification procedure is also suitable for purifying the VEGF mutant polypeptides obtained from chemical synthesis.

i. Solubility Fractionation

Often as an initial step, and if the protein mixture is complex, an initial salt fractionation can separate many of the unwanted host cell proteins (or proteins derived from the cell culture media) from the recombinant protein of interest, e.g., a VEGF mutant polypeptide of the present invention. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol is to add saturated ammonium sulfate to a protein solution so that the resultant ammonium sulfate concentration is between 20-30%. This will precipitate the most hydrophobic proteins. The precipitate is discarded (unless the protein of interest is hydrophobic) and ammonium sulfate is added to the supernatant to a concentration known to precipitate the protein of interest. The precipitate is then solubilized in buffer and the excess salt removed if necessary, through either dialysis or diafiltration. Other methods that rely on solubility of proteins, such as cold ethanol precipitation, are well known to those of skill in the art and can be used to fractionate complex protein mixtures.

ii. Size Differential Filtration

Based on a calculated molecular weight, a protein of greater and lesser size can be isolated using ultrafiltration through membranes of different pore sizes (for example, Amicon or Millipore membranes). As a first step, the protein mixture is ultrafiltered through a membrane with a pore size that has a lower molecular weight cut-off than the molecular weight of a protein of interest, e.g., a VEGF mutant polypeptide. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

iii. Column Chromatography

The proteins of interest (such as a VEGF mutant polypeptide of the present invention) can also be separated from other proteins on the basis of their size, net surface charge, hydrophobicity, or affinity for ligands. In addition, antibodies raised against a segment of VEGF165 (e.g., a segment outside of the integrin-binding domain, the 111-165 segment of SEQ ID NO: 1) can be conjugated to column matrices and the VEGF mutant polypeptide immunopurified. All of these methods are well-known in the art.

It will be apparent to one of skill that chromatographic techniques can be performed at any scale and using equipment from many different manufacturers (e.g., Pharmacia Biotech).

### IV. Identification of Inhibitors for VEGF165-Integrin Binding

### A. VEGF165-Integrin Binding Assays

An in vitro assay can be used to detect VEGF-integrin binding and to identify compounds that are capable of inhibiting VEGF-integrin binding. In general, such an assay can be performed in the presence of a VEGF165, such as human VEGF165 or its 111-165 segment, and an integrin, such as αvβ3, that are known to bind each other, under conditions permitting such binding. For convenience, one of the binding partners may be immobilized onto a solid support and/or labeled with a detectable moiety. A third molecule, such as an antibody (which may include a detectable label) to one of the binding partners, can also be used to facilitate detection.

In some cases, the binding assays can be performed in a cell-free environment; whereas in other cases, the binding assays can be performed on cell surface, frequently using cells recombinantly or endogenously expressing an appropriate integrin molecule. More details and some examples of such binding assays can be found in the Examples section of this application.

To screen for compounds capable of inhibiting VEGF-integrin binding, the above-described assays are performed both in the presence and absence of a test compound, the level of binding between a polypeptide comprising the 111-165 segment of SEQ ID NO:1 (or the full-length VEGF165) and integrin αvβ3 is then compared. If the binding level is suppressed at the presence of the test compound at a level of at least 10%, more preferably at least 20%, 30%, 40%, or 50%, or even higher, the test compound is then deemed an inhibitor of VEGF-integrin binding and may be subject to further testing to confirm its ability to inhibit VEGF signaling. Conversely, if the binding level is increased at the presence of the test compound at a level of at least 10%, more preferably at least 20%, 30%, 40%, 50%, 100%, 200%, or even higher, the test compound is then deemed an enhancer of VEGF-integrin binding and may be subject to further testing to confirm its ability to promote VEGF signaling.

The binding assay is also useful for determining whether or not a polypeptide derived from a wild-type VEGF165 protein can effectively and specifically bind integrin. For instance, a polypeptide comprising the amino acid sequence of a VEGF165 protein with one or more point mutations (e.g., at one or more residues 123, 124, 125, 140, 145, and 149) may be recombinantly expressed, purified, and placed in a binding assay with integrin αvβ3, substituting a full length wild type VEGF165 protein, which may be used in a control assay to provide a comparison basis. If deemed to have sufficient integrin-binding ability, a polypeptide comprising a VEGF-integrin binding sequence (i.e., the 111-165 segment of SEQ ID NO: 1) can then be used, in place of a wild-type full length VEGF165 protein, in a binding assay for identifying inhibitors of VEGF-integrin binding. Conversely, a VEGF mutant that has lost or greatly diminished its integrin-binding capability may be further tested for its dominant negative features. Similarly, a polypeptide comprising a core sequence with a high level of homology (e.g., 90%, 95%, 97%, 98%, 99% or higher) to the sequence of a wild-type VEGF165 protein can be tested and, if appropriate, can be used, in place of a wild-type full length VEGF165 protein, in a binding assay for identifying inhibitors of VEGF-integrin binding. Such a variant of VEGF165 protein may also be tested for its potential dominant negative features and utilities.

Inhibitors of VEGF-integrin binding or VEGF signaling can have diverse chemical and structural features. For instance, an inhibitor can be a non-functional VEGF mutant that retaining KDR-binding ability, an antibody to either VEGF165 or integrin that interferes with VEGF-integrin binding, or any small molecule or macromolecule that simply hinders the interaction between VEGF165 and integrin. Essentially any chemical compound can be tested as a potential inhibitor of VEGF-integrin binding. Most preferred are generally compounds that can be dissolved in aqueous or organic (especially DMSO-based) solutions. Inhibitors can be identified by screening a combinatorial library containing a large number of potentially effective compounds. Such combinatorial chemical libraries can be screened in one or more assays, as described herein, to identify those library members (particular chemical species or subclasses) that display a desired characteristic activity. The compounds thus identified can serve as conventional “lead compounds” or can themselves be used as potential or actual therapeutics.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)) and carbohydrate libraries (see, e.g., Liang et al., Science, 274:1520-1522 (1996) and U.S. Pat. No. 5,593,853). Other chemistries for generating chemical diversity libraries can also be used. Such chemistries include, but are not limited to: peptoids (PCT Publication No. WO 91/19735), encoded peptides (PCT Publication WO 93/20242), random bio-oligomers (PCT Publication No. WO 92/00091), benzodiazepines (U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with β-D-glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see, Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), small organic molecule libraries (see, e.g., benzodiazepines, Baum C&EN, Jan 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; and benzodiazepines, U.S. Pat. No. 5,288,514).

### B. VEGF165 Signaling Assays

The inhibitors of VEGF-integrin binding are useful for their ability to inhibit VEGF165 signaling, especially as anti-inflammation or anti-cancer therapeutics. Assays for confirming such inhibitory effect of an inhibitor can be performed in vitro or in vivo. An in vitro assay typically involves exposure of cultured cells to an inhibitor and monitoring of subsequent biological and biochemical changes in the cells. For example, following exposure to 0.1-20 μg/ml an inhibitor for 0.5-48 hours, suitable cells (such as those expressing integrin αvβ3 and KDR) are examined for their proliferation/survival status using methods such as direct cell number counting, BrdU or H³-thymidine incorporation, tetrazolium salt 3,[4,5-dimethylthiazol-2-yl]-2,5-diphenyltetrazolium bromide (MTT) cell proliferation assay, 3-(4,5-dimethylthiazol-2-yl)-5-(3-carboxymethoxyphenyl)-2-(4-sulfophenyl)-2H-tetrazolium (MTS) cell proliferation assay, chicken embryo allantoic membrane (CAM) assay, TUNNEL assay, annexin V binding assay, etc. Further downstream changes due to VEGF165 signaling, e.g., changes in ERK1/2 activation induced by wild-type VEGF165, can also be monitored to provide an indication of suppressed VEGF165 signaling. In addition, tumorigenicity of cancer cells is useful parameters for monitoring and can be tested by methods such as colony formation assays or soft agar assays. Detailed description of some exemplary assays can be found in the Examples section of this disclosure. An inhibitory effect is detected when a decrease in VEGF165 signaling, as indicated by any one aforementioned parameter, of at least 10%, 20%, 30%, 40%, 50%, 60%, 70%, 80%, 90% or more is observed.

The effects of a VEGF-integrin binding inhibitor of the present invention can also be demonstrated in in vivo assays. For example, an inhibitor of VEGF-integrin binding can be injected into animals that have a compromised immune system (e.g., nude mice, SCID mice, or NOD/SCID mice) and therefore permit xenograft tumors. Injection methods can be intravenous, intraperitoneal, or intratumoral in nature. Tumor development is subsequently monitored by various means, such as measuring tumor volume and scoring secondary lesions due to metastases, in comparison with a control group of animals with similar tumors but not given the inhibitors. Similarly, in vivo assays can be performed in an inflammation animal model to test and verify the capability of a VEGF mutant in inhibiting inflammatory response induced by VEGF-integrin signaling. An inhibitory effect is detected when a negative effect on tumor growth or metastasis is established in the test group. Preferably, the negative effect is at least a 10% decrease; more preferably, the decrease is at least 20%, 30%, 40%, 50%, 60%, 70%, 80%, or 90%.

### V. Pharmaceutical Compositions and Administration

The present invention also provides pharmaceutical compositions or physiological compositions comprising an effective amount of a compound that inhibits VEGF-integrin binding, such as a functionally dominant negative VEGF165 mutant having one or more mutations within the 111-165 segment, e.g., R123A/R124A/K125A/K140A/R145A/R149A, R123A/R124A/K125A, K140A/R145A/R149A of SEQ ID NO:1, or its encoding nucleic acid, inhibiting VEGF165 signaling in both prophylactic and therapeutic applications. Such pharmaceutical or physiological compositions also include one or more pharmaceutically or physiologically acceptable excipients or carriers. Pharmaceutical compositions of the invention are suitable for use in a variety of drug delivery systems. Suitable formulations for use in the present invention are found in Remington's Pharmaceutical Sciences, Mack Publishing Company, Philadelphia, PA, 17th ed. (1985). For a brief review of methods for drug delivery, see, Langer, Science 249: 1527-1533 (1990).

The pharmaceutical compositions of the present invention can be administered by various routes, e.g., oral, nasal, subcutaneous, transdermal, intramuscular, intravenous, or intraperitoneal. The preferred routes of administering the pharmaceutical compositions are local delivery to an organ or tissue suffering from a condition exacerbated by over-activation of VEGF-integrin signaling (e.g., intratumoral injection to a tumor) at daily doses of about 0.01-5000 mg, preferably 5-500 mg, of a VEGF-integrin binding inhibitor for a 70 kg adult human per day. The appropriate dose may be administered in a single daily dose or as divided doses presented at appropriate intervals, for example as two, three, four, or more subdoses per day.

For preparing pharmaceutical compositions containing a VEGF-integrin binding inhibitor, inert and pharmaceutically acceptable carriers are used. The pharmaceutical carrier can be either solid or liquid. Solid form preparations include, for example, powders, tablets, dispersible granules, capsules, cachets, and suppositories. A solid carrier can be one or more substances that can also act as diluents, flavoring agents, solubilizers, lubricants, suspending agents, binders, or tablet disintegrating agents; it can also be an encapsulating material.

In powders, the carrier is generally a finely divided solid that is in a mixture with the finely divided active component, e.g., a VEGF165 dominant negative mutant polypeptide. In tablets, the active ingredient (e.g., an inhibitor of VEGF-integrin binding such as a VEGF mutant polypeptide) is mixed with the carrier having the necessary binding properties in suitable proportions and compacted in the shape and size desired.

For preparing pharmaceutical compositions in the form of suppositories, a low-melting wax such as a mixture of fatty acid glycerides and cocoa butter is first melted and the active ingredient is dispersed therein by, for example, stirring. The molten homogeneous mixture is then poured into convenient-sized molds and allowed to cool and solidify.

Powders and tablets preferably contain between about 50% to about 70% by weight of the active ingredient of an inhibitor of VEGF-integrin binding (such as a VEGF mutant polypeptide). Suitable carriers include, for example, magnesium carbonate, magnesium stearate, talc, lactose, sugar, pectin, dextrin, starch, tragacanth, methyl cellulose, sodium carboxymethyl cellulose, a low-melting wax, cocoa butter, and the like.

The pharmaceutical compositions can include the formulation of the active compound of a VEGF-integrin binding inhibitor (such as a VEGF mutant polypeptide) with encapsulating material as a carrier providing a capsule in which the inhibitor (with or without other carriers) is surrounded by the carrier, such that the carrier is thus in association with the compound. In a similar manner, cachets can also be included. Tablets, powders, cachets, and capsules can be used as solid dosage forms suitable for oral administration.

Liquid pharmaceutical compositions include, for example, solutions suitable for oral or parenteral administration, suspensions, and emulsions suitable for oral administration. Sterile water solutions of the active component (e.g., a dominant-negative VEGF mutant polypeptide) or sterile solutions of the active component in solvents comprising water, buffered water, saline, PBS, ethanol, or propylene glycol are examples of liquid compositions suitable for parenteral administration. The compositions may contain pharmaceutically acceptable auxiliary substances as required to approximate physiological conditions, such as pH adjusting and buffering agents, tonicity adjusting agents, wetting agents, detergents, and the like.

Sterile solutions can be prepared by dissolving the active component (e.g., a VEGF-integrin binding inhibitor such as a VEGF mutant polypeptide) in the desired solvent system, and then passing the resulting solution through a membrane filter to sterilize it or, alternatively, by dissolving the sterile compound in a previously sterilized solvent under sterile conditions. The resulting aqueous solutions may be packaged for use as is, or lyophilized, the lyophilized preparation being combined with a sterile aqueous carrier prior to administration. The pH of the preparations typically will be between 3 and 11, more preferably from 5 to 9, and most preferably from 7 to 8.

The pharmaceutical compositions containing a VEGF-integrin binding inhibitor (such as a VEGF mutant polypeptide) can be administered for prophylactic and/or therapeutic treatments. In therapeutic applications, compositions are administered to a patient already suffering from a condition that may be exacerbated by over-activation of VEGF-integrin signaling in an amount sufficient to prevent, cure, reverse, or at least partially slow or arrest the symptoms of the condition and its complications. An amount adequate to accomplish this is defined as a “therapeutically effective dose.” Amounts effective for this use will depend on the severity of the disease or condition and the weight and general state of the patient, but generally range from about 0.1 mg to about 2,000 mg of the inhibitor per day for a 70 kg patient, with dosages of from about 5 mg to about 500 mg of the inhibitor per day for a 70 kg patient being more commonly used.

In prophylactic applications, pharmaceutical compositions containing VEGF165-integrin binding inhibitors (such as a VEGF mutant polypeptide) are administered to a patient susceptible to or otherwise at risk of developing a disease or condition in which undesirable over-activation of VEGF165-integrin signaling is present, in an amount sufficient to delay or prevent the onset of the symptoms. Such an amount is defined to be a “prophylactically effective dose.” In this use, the precise amounts of the inhibitor again depend on the patient's state of health and weight, but generally range from about 0.1 mg to about 2,000 mg of the inhibitor for a 70 kg patient per day, more commonly from about 5 mg to about 500 mg for a 70 kg patient per day.

Single or multiple administrations of the compositions can be carried out with dose levels and pattern being selected by the treating physician. In any event, the pharmaceutical formulations should provide a quantity of a VEGF-integrin binding sufficient to effectively inhibit VEGF signaling in the patient, either therapeutically or prophylactically.

### VI. Therapeutic Applications Using Nucleic Acids

A variety of diseases can be treated by therapeutic approaches that involve introducing a nucleic acid encoding a polypeptide inhibitor of integrin-VEGF165 binding (such as a VEGF mutant polypeptide) into a cell such that the coding sequence is transcribed and the polypeptide inhibitor is produced in the cell. Diseases amenable to treatment by this approach include a broad spectrum of inflammatory diseases and disorders as well as cancers, the survival, growth, and metastasis of which rely on to some extent the continued signaling of VEGF165 or integrin family members. For discussions on the application of gene therapy towards the treatment of genetic as well as acquired diseases, see, Miller Nature 357:455-460 (1992); and Mulligan Science 260:926-932 (1993).

### A. Vectors for Gene Delivery

For delivery to a cell or organism, a polynucleotide encoding a polypeptide that inhibits VEGF165-integrin binding (e.g., dominant-negative mutant R123A/R124A/K125A, K140A/R145A/R149A, or R123A/R124A/K125A/K140A/R145A/R149A of SEQ ID NO:1) can be incorporated into a vector. Examples of vectors used for such purposes include expression plasmids capable of directing the expression of the nucleic acids in the target cell. In other instances, the vector is a viral vector system wherein the polynucleotide is incorporated into a viral genome that is capable of transfecting the target cell. In a preferred embodiment, the polynucleotide encoding a polypeptide inhibitor can be operably linked to expression and control sequences that can direct expression of the polypeptide in the desired target host cells. Thus, one can achieve expression of the polypeptide inhibitor under appropriate conditions in the target cell.

### B. Gene Delivery Systems

Viral vector systems useful in the expression of a polypeptide inhibitor of VEGF-integrin binding (such as a VEGF mutant polypeptide) include, for example, naturally occurring or recombinant viral vector systems. Depending upon the particular application, suitable viral vectors include replication competent, replication deficient, and conditionally replicating viral vectors. For example, viral vectors can be derived from the genome of human or bovine adenoviruses, vaccinia virus, herpes virus, adeno-associated virus, minute virus of mice (MVM), HIV, sindbis virus, and retroviruses (including but not limited to Rous sarcoma virus), and MoMLV. Typically, the genes of interest (e.g., one encoding for a polypeptide inhibitor of the present invention) are inserted into such vectors to allow packaging of the gene construct, typically with accompanying viral DNA, followed by infection of a sensitive host cell and expression of the gene of interest.

As used herein, “gene delivery system” refers to any means for the delivery of a nucleic acid of the invention to a target cell. In some embodiments of the invention, nucleic acids are conjugated to a cell receptor ligand for facilitated uptake (e.g., invagination of coated pits and internalization of the endosome) through an appropriate linking moiety, such as a DNA linking moiety (Wu et al., J. Biol. Chem. 263:14621-14624 (1988); WO 92/06180). For example, nucleic acids can be linked through a polylysine moiety to asialo-oromucocid, which is a ligand for the asialoglycoprotein receptor of hepatocytes.

Similarly, viral envelopes used for packaging gene constructs that include the nucleic acids of the invention can be modified by the addition of receptor ligands or antibodies specific for a receptor to permit receptor-mediated endocytosis into specific cells (see, e.g., WO 93/20221, WO 93/14188, and WO 94/06923). In some embodiments of the invention, the DNA constructs of the invention are linked to viral proteins, such as adenovirus particles, to facilitate endocytosis (Curiel et al., Proc. Natl. Acad. Sci. U.S.A. 88:8850-8854 (1991)). In other embodiments, molecular conjugates of the instant invention can include microtubule inhibitors (WO/9406922), synthetic peptides mimicking influenza virus hemagglutinin (Plank et al., J. Biol. Chem. 269:12918-12924 (1994)), and nuclear localization signals such as SV40 T antigen (WO93/19768).

Retroviral vectors may also be useful for introducing the coding sequence of a polypeptide inhibitor of the invention into target cells or organisms. Retroviral vectors are produced by genetically manipulating retroviruses. The viral genome of retroviruses is RNA. Upon infection, this genomic RNA is reverse transcribed into a DNA copy which is integrated into the chromosomal DNA of transduced cells with a high degree of stability and efficiency. The integrated DNA copy is referred to as a provirus and is inherited by daughter cells as is any other gene. The wild type retroviral genome and the proviral DNA have three genes: the gag, the pol and the env genes, which are flanked by two long terminal repeat (LTR) sequences. The gag gene encodes the internal structural (nucleocapsid) proteins; the pol gene encodes the RNA directed DNA polymerase (reverse transcriptase); and the env gene encodes viral envelope glycoproteins. The 5′ and 3′ LTRs serve to promote transcription and polyadenylation of virion RNAs. Adjacent to the 5′ LTR are sequences necessary for reverse transcription of the genome (the tRNA primer binding site) and for efficient encapsulation of viral RNA into particles (the Psi site) (see, Mulligan, In: Experimental Manipulation of Gene Expression, Inouye (ed), 155-173 (1983); Mann et al., Cell 33:153-159 (1983); Cone and Mulligan, Proceedings of the National Academy of Sciences, U.S.A., 81:6349-6353 (1984)).

The design of retroviral vectors is well known to those of ordinary skill in the art. In brief, if the sequences necessary for encapsidation (or packaging of retroviral RNA into infectious virions) are missing from the viral genome, the result is a cis acting defect which prevents encapsidation of genomic RNA. However, the resulting mutant is still capable of directing the synthesis of all virion proteins. Retroviral genomes from which these sequences have been deleted, as well as cell lines containing the mutant genome stably integrated into the chromosome are well known in the art and are used to construct retroviral vectors. Preparation of retroviral vectors and their uses are described in many publications including, e.g., European Patent Application EPA 0 178 220; U.S. Pat. No. 4,405,712, Gilboa Biotechniques 4:504-512 (1986); Mann et al., Cell 33:153-159 (1983); Cone and Mulligan Proc. Natl. Acad. Sci. USA 81:6349-6353 (1984); Eglitis et al. Biotechniques 6:608-614 (1988); Miller et al. Biotechniques 7:981-990 (1989); Miller (1992) supra; Mulligan (1993), supra; and WO 92/07943.

The retroviral vector particles are prepared by recombinantly inserting the desired nucleotide sequence into a retrovirus vector and packaging the vector with retroviral capsid proteins by use of a packaging cell line. The resultant retroviral vector particle is incapable of replication in the host cell but is capable of integrating into the host cell genome as a proviral sequence containing the desired nucleotide sequence. As a result, the patient is capable of producing, for example, a polypeptide or polynucleotide of the invention.

Packaging cell lines that are used to prepare the retroviral vector particles are typically recombinant mammalian tissue culture cell lines that produce the necessary viral structural proteins required for packaging, but which are incapable of producing infectious virions. The defective retroviral vectors that are used, on the other hand, lack these structural genes but encode the remaining proteins necessary for packaging. To prepare a packaging cell line, one can construct an infectious clone of a desired retrovirus in which the packaging site has been deleted. Cells comprising this construct will express all structural viral proteins, but the introduced DNA will be incapable of being packaged. Alternatively, packaging cell lines can be produced by transforming a cell line with one or more expression plasmids encoding the appropriate core and envelope proteins. In these cells, the gag, pol, and env genes can be derived from the same or different retroviruses.

A number of packaging cell lines suitable for the present invention are also available in the prior art. Examples of these cell lines include Crip, GPE86, PA317 and PG13 (see Miller et al., J. Virol. 65:2220-2224 (1991)). Examples of other packaging cell lines are described in Cone and Mulligan Proceedings of the National Academy of Sciences, USA, 81:6349-6353 (1984); Danos and Mulligan Proceedings of the National Academy of Sciences, USA, 85:6460-6464 (1988); Eglitis et al. (1988), supra; and Miller (1990), supra.

Packaging cell lines capable of producing retroviral vector particles with chimeric envelope proteins may be used. Alternatively, amphotropic or xenotropic envelope proteins, such as those produced by PA317 and GPX packaging cell lines may be used to package the retroviral vectors.

### C. Pharmaceutical Formulations

When used for pharmaceutical purposes, the nucleic acid encoding a VEGF-integrin binding inhibitor polypeptide (such as a VEGF mutant polypeptide) is generally formulated in a suitable buffer, which can be any pharmaceutically acceptable buffer, such as phosphate buffered saline or sodium phosphate/sodium sulfate, Tris buffer, glycine buffer, sterile water, and other buffers known to the ordinarily skilled artisan such as those described by Good et al. Biochemistry 5:467 (1966).

The compositions can additionally include a stabilizer, enhancer or other pharmaceutically acceptable carriers or vehicles. A pharmaceutically acceptable carrier can contain a physiologically acceptable compound that acts, for example, to stabilize the nucleic acids of the invention and any associated vector. A physiologically acceptable compound can include, for example, carbohydrates, such as glucose, sucrose or dextrans, antioxidants, such as ascorbic acid or glutathione, chelating agents, low molecular weight proteins or other stabilizers or excipients. Other physiologically acceptable compounds include wetting agents, emulsifying agents, dispersing agents or preservatives, which are particularly useful for preventing the growth or action of microorganisms. Various preservatives are well known and include, for example, phenol and ascorbic acid. Examples of carriers, stabilizers or adjuvants can be found in Remington's Pharmaceutical Sciences, Mack Publishing Company, Philadelphia, PA, 17th ed. (1985).

### D. Administration of Formulations

The formulations containing a nucleic acid encoding a polypeptide inhibitor of the binding between VEGF165 and integrin can be delivered to any tissue or organ using any delivery method known to the ordinarily skilled artisan. In some embodiments of the invention, the nucleic acids encoding the inhibitor polypeptides are formulated for systemic administration such as oral or nasal administration or via injection, e.g., intravenous, intraperitoneal, subcutaneous, or intramuscular injection, for local delivery such as topical application, delivery by suppository, or intratumoral injection.

The formulations containing the nucleic acid of the invention are typically administered to a cell. The cell can be provided as part of a tissue, such as an epithelial membrane, or as an isolated cell, such as in tissue culture. The cell can be provided in vivo, ex vivo, or in vitro.

The formulations can be introduced into the tissue of interest in vivo or ex vivo by a variety of methods. In some embodiments of the invention, the nucleic acids of the invention are introduced into cells by such methods as microinjection, calcium phosphate precipitation, liposome fusion, ultrasound, electroporation, or biolistics. In further embodiments, the nucleic acids are taken up directly by the tissue of interest.

In some embodiments of the invention, the nucleic acids of the invention are administered ex vivo to cells or tissues explanted from a patient, then returned to the patient. Examples of ex vivo administration of therapeutic gene constructs include Nolta et al., Proc Natl. Acad. Sci. USA 93(6):2414-9 (1996); Koc et al., Seminars in Oncology 23(1):46-65 (1996); Raper et al., Annals of Surgery 223(2):116-26 (1996); Dalesandro et al., J. Thorac. Cardi. Surg., 11(2):416-22 (1996); and Makarov et al., Proc. Natl. Acad. Sci. USA 93(1):402-6 (1996).

Effective dosage of the formulations will vary depending on many different factors, including means of administration, target site, physiological state of the patient, and other medicines administered. Thus, treatment dosages will need to be titrated to optimize safety and efficacy. In determining the effective amount of the vector to be administered, the physician should evaluate the particular nucleic acid used, the disease state being diagnosed; the age, weight, and overall condition of the patient, circulating plasma levels, vector toxicities, progression of the disease, and the production of anti-vector antibodies. The size of the dose also will be determined by the existence, nature, and extent of any adverse side-effects that accompany the administration of a particular vector. To practice the present invention, doses ranging from about 10 ng-1 g, 100 ng-100 mg, 1 μg-10 mg, or 30-300 μg DNA per patient are typical. Doses generally range between about 0.01 and about 50 mg per kilogram of body weight, preferably between about 0.1 and about 5 mg/kg of body weight or about 10⁸-10¹⁰ or 10¹² particles per injection. In general, the dose equivalent of a naked nucleic acid from a vector is from about 1 μg-100 μg for a typical 70 kg patient, and doses of vectors which include a retroviral particle are calculated to yield an equivalent amount of nucleic acid encoding a polypeptide that inhibits the binding between integrin αvβ3 and human VEGF165.

### VII. Kits

The invention also provides kits for suppressing inflammation and angiogenesis by inhibiting VEGF165 signaling according to the method of the present invention. The kits typically include a container that contains a pharmaceutical composition having an effective amount of an inhibitor of VEGF-integrin binding (such as a dominant-negative mutant VEGF polypeptide or a polynucleotide sequence encoding the polypeptide) as well as informational material containing instructions on how to dispense the pharmaceutical composition, including description of the type of patients who may be treated (e.g., patients with an inflammatory condition or cancer with over-activated VEGF-integrin signaling and thus excessive or inappropriate angiogenesis), the schedule (e.g., dose and frequency) and route of administration, and the like.

### EXAMPLES

The following examples are provided by way of illustration only and not by way of limitation. Those of skill in the art will readily recognize a variety of non-critical parameters that could be changed or modified to yield essentially the same or similar results.

### Example 1

VEGF-A is a key cytokine in tumor angiogenesis and a major therapeutic target for cancer. VEGF165 is the predominant isoform and is the most potent angiogenesis stimulant. VEGFR2/KDR domains 2 and 3 (D2D3) bind to the N-terminal domain (NTD, residues 1-110) of VEGF165. Since removal of the heparin-binding domain (HBD, residues 111-165) markedly reduced the mitogenic activity of VEGF165, it has been proposed that the HBD plays a critical role in the mitogenicity of VEGF165. Integrin αvβ3 has been shown to bind to VEGF165, but the role of integrin αvβ3 in VEGF165 signaling are unclear. Here we describe that αvβ3 specifically bound to the isolated HBD, but not to the NTD. We identified several critical amino acid residues in HBD for integrin binding (Arg-123, Arg-124, Lys-125, Lys-140, Arg-145, and Arg-149) by docking simulation and mutagenesis, and generated full-length VEGF165 that is defective in integrin binding by including mutations in the HBD. The full-length VEGF165 mutant defective in integrin binding (R123A/R124A/K125A/K140A/R145A/R149A) was defective in ERK1/2 phosphorylation, integrin 03 phosphorylation, and KDR phosphorylation, although the mutation did not affect KDR binding to VEGF165. We propose a model in which VEGF165 induces KDR (through NTD)-VEGF165 (through HBD)-integrin αvβ3 ternary complex formation on the cell surface and this process is critically involved in potent mitogenicity of VEGF165.

### Introduction

It has been proposed that growth factor signaling requires integrins for cell responses to growth factor ligation of their cognate cell surface receptors [1, 2]. The specific mechanisms and the extent of this integrin-growth factor crosstalk are still not been established. Integrins are cell surface heterodimers that act as receptors for extracellular matrix ligands, cell-surface ligands (ICAM-1, VCAM-1), and soluble ligands that include growth factors [1, 3]. The finding that integrin αvβ3 antagonists inhibit fibroblast growth factor-2 (basic FGF, FGF2) signaling [4] suggested that αvβ3 is involved in growth factor signaling through a crosstalk mechanism [5-7]. We previously reported that FGF1 and FGF2 directly bind to integrin αvβ3, leading to formation of an integrin-FGF-FGFR ternary complex that is required for FGF signaling functions (the ternary complex model) [8-11]. We showed that this model can be applied to other growth factors as well, including IGF1, IGF2, neuregulin-1, fractalkine, and CD40L [12-16]. Thus, direct binding of integrin αvβ3 to growth factors is required for their signaling functions. Notably, we showed that growth factor mutants defective in integrin binding are also defective in signaling functions and can act as antagonists of the signaling process, while the mutants still bind to their cognate receptors [8-11, 13, 14, 16].

Vascular endothelial growth factor (VEGF-A) is a key cytokine in angiogenesis and a major therapeutic target. It has been proposed that VEGF-A signaling requires integrin αvβ3, but it is unclear whether the ternary complex model can be applied, since VEGF-A binding to αvβ3 has not been documented. Among the six main isoforms of VEGF-A, VEGF165 (the 165-amino-acid protein) is the predominant gene product in human tissues and the most potent angiogenesis stimulant [17, 18]. VEGF165 forms homodimers of two anti-parallel monomers that interact via their N-terminal domains which harbor the cognate receptor binding site. VEGF165 exerts mitogenicity by binding to the VEGFR1 (FLT1) and VEGFR2 (KDR) receptor tyrosine kinases [19]. KDR, a 230 kDa glycoprotein, has a lower affinity for VEGF165 (KD=0.75-2×10⁻¹⁰ M) than VEGFR1 (KD 1-2×10⁻¹¹ M). Yet, KDR is the primary mediator of VEGF165 signaling [20]. Within its N-terminal region, KDR has seven IgG-like extracellular domains, of which domains 2 (D2) and 3 (D3) interact with the N-terminal domain of VEGF165 with high affinities [20]. The clinically used anti-angiogenic monoclonal antibody Avastin (Bevacizumab), binds to the N-terminal domain of VEGF165 (residues 1-110) and inhibits VEGF165 binding to KDR. The C-terminal domain of VEGF165 encompasses a heparin-binding domain (HBD, residues 111-165) and a neuropilin-binding site. A plasmin-cleavage site located between the ligand's N-terminal and heparin binding domains enables proteolytic removal of the HBD, which markedly reduces the mitogenicity of VEGF165 in endothelial cells [21]. This indicates that KDR binding to the N-terminal VEGF165 domain may not be sufficient to exert a cell growth response when the HBD is missing. The reduced mitogenic activity of the VEGF165 homodimer which lacks the HBD is similar to that observed for VEGF121, a splice variant that lacks exons 6a and 7 which encode most of the HBD. Although evidence suggests that the HBD plays a role in VEGF165 dependent processes including angiogenesis, it is unclear how the HBD contributes to VEGF165 mediated cell responses.

In the present study we first showed that αvβ3 directly binds to HBD. We then identified amino acid residues critical for αvβ3 by docking simulation of interaction between HBD and αvβ3, and subsequent mutagenesis studies. Our results indicate that integrin αvβ3 binds to the HBD of VEGF165 and leads to formation of a ternary complex between αvβ3, VEGF165 and KDR. Having identified the integrin binding site within the VEGF165-HBD, our studies using VEGF165-HBD mutants defective in integrin binding suggest that αvβ3 binding to VEGF165 is essential for KDR-mediated VEGF165 cell activation. The results from our work enable new synergistic therapies that co-target αvβ3- and KDR-VEGF interaction to control diseases that involve aberrant cell proliferation and angiogenesis.

### Materials and Methods

Recombinant soluble αvβ3 was synthesized in Chinese hamster ovary (CHO) K1 cells using the soluble αv and β3 expression constructs and purified by nickel-nitrilotriacetic acid (Ni-NTA) affinity chromatography as described [22].

### Plasmid Construction and Protein Purification

We did PCR amplification of cDNA fragments encoding the N-terminal domain, the HBD, and full length VEGF. More precisely, The cDNA fragments encoding the N-terminal domain, the HBD, and full length VEGF were amplified by PCR using full-length VEGF cDNA (Open Biosystems, Lafayette, CO) as a template, and were subcloned into the BamHI/EcoRI site of the PET28a expression vector. The insoluble protein was synthesized in BL21 induced by IPTG and was solubilized in 8 M urea, purified by Ni-NTA affinity chromatography and refolded as described [12].

### ELISA-Type Binding Assays

A 96-well plate was coated with PBS-diluted protein and incubated at 37° C. for one hour. The wells were further blocked with 0.1% BSA/PBS boiled at 80° C. for 20 min then cooled down before application of 300 μl/well. After half an hour of incubation at room temperature, soluble αvβ3 in Hepes-Tyrode buffer+1 mM MnCl₂ was added, and the plate was further incubated at room temperature for one hour. Bound integrin αvβ3 was quantified using mouse monoclonal anti-integrin β3 (AV10) and HRP-conjugated anti-mouse IgG and peroxidase substrate.

### Surface Plasmon Resonance Study

Soluble αvβ3 was immobilized on the CM5 sensor chip using a standard amine coupling procedure in HBS-EP buffer (0.01 M Hepes, pH 7.4, 0.15 M NaCl, 1 mM Mn²⁺, and 0.0005% of surfactant P20), and the same buffer. Samples were injected at 50 μl/min for 1.8 min. The HBS-EP buffer was then injected at 50 μl/min for 3 min to allow the bound VEGFs to dissociate from αvβ3.

### Cell Adhesion to VEGF165

A 96-well plate was coated with PBS-diluted protein and incubated at 37° C. for one hour. The wells were further blocked with 0.1% BSA/PBS boiled at 80° C. for 20 min then cooled down before application of 300 μl/well. After half an hour of incubation at room temperature, 1×10⁵ cells/well in DMEM or Hepes-Tyrode buffer+1 mM MgCl₂/1 mM MnCl₂ were added and the plate was further incubated in a 5% CO₂ containing incubator at 37° C. for one hour. Cells were removed and washed with dilution buffer A Phosphatase buffer (100 l/well) was applied, and the plate was incubated at 37° C. for 45 min. 1 M NaOH (50 l/well) was applied to stop the reaction and the plate was read at OD=405 nm by the plate reader.

### Docking Simulation

Docking simulation of interaction between the HBD of VEGF165 (2VGH.pdb) and integrin αvβ3 was performed using AutoDock3 as described [12]. In the present study, we used the headpiece (residues 1-438 of αv and residues 55-432 of β3) of αvβ3 (open-headpiece form, 1L5G.pdb). Cations were not present in αvβ3 during docking simulation [8, 23].

### SDS-PAGE Analysis of Wild Type and Mutant VEGF165

In mutant VEGF165 all six amino acids within the HBD identified as required for integrin αvβ3 binding were changed to Ala (R123A/R124A/K125A/K140A/R145A/R149A). Molecular size values in kDa.

### Pull-Down of VEGF165 by KDR

His-tagged KDR was immobilized on Ni-NTR beads and incubated with full-length VEGF165 wild type (WT) vs VEGF165 mutant (mut) protein in binding buffer for 2 h at 4° C., before elution and analysis of the retained protein by SDS-PAGE. Both wild-type and mutant VEGF165 bind to KDR.

### ERK1/2 Activation in HUVECs by VEGF165

Starved HUVECs in M200 basal medium without low serum growth supplement were treated with VEGF165 (10 ng/ml) for 10 min before lysis and Western blot analysis. Wild-type (WT) but not mutant (mut) VEGF165 induces ERK1/2 phosphorylation in human umbilical vein endothelial cells (HUVEC).

Integrin αvβ3 Phosphorylation in HUVEC in Response to VEGF165

Starved HUVECs were treated with VEGF165 wild type vs mutant (10 ng/ml) before lysis and Western blot analysis of β3 integrin subunit phosphorylation. Wild-type (WT) but not mutant (mut) VEGF165 induces integrin αvβ3 phosphorylation in HUVECs.

### KDR Y1175 Phosphorylation in HUVEC in Response to VEGF165

In HUVECs, wild type (WT) but not mutant (mut) VEGF165 activates KDR Y1175 phosphorylation known to stimulate endothelial cell proliferation and migration. Starved HUVECs were treated for Western blot analysis.

### Statistical Analysis

We reported results as the mean±standard error of the mean and performed calculations using Prism 7. Statistical analysis was performed using ANOVA.

### Results

Direct Binding of Integrin αvβ3 to the VEGF165 Heparin Binding Domain (HBD)

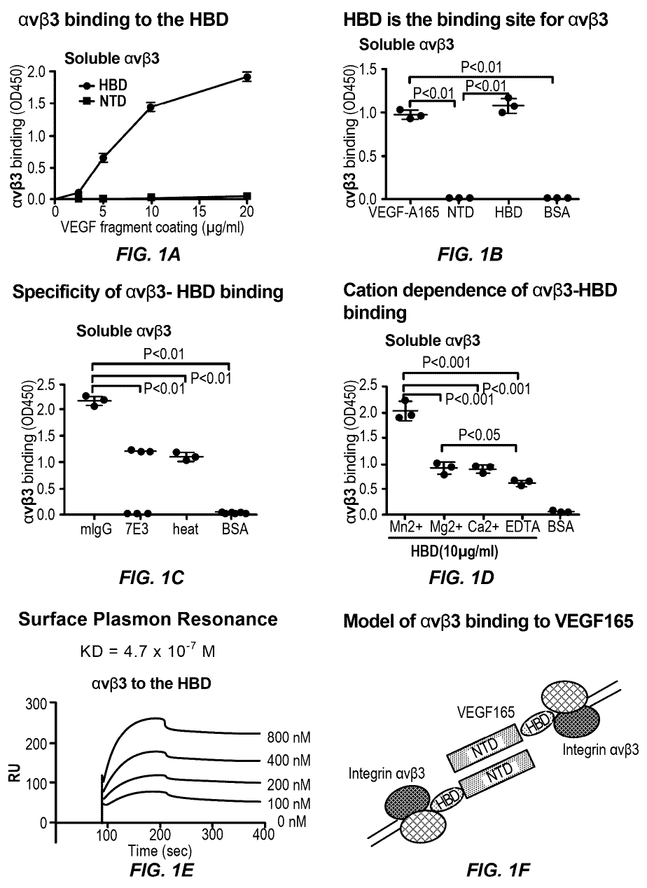

It has been proposed that VEGF165 interacts with αvβ3, but VEGF121 which lacks the HBD, does not [24]. This implies that the HBD likely contains a binding site for αvβ3, however, the specifics of an HBD-αvβ3 interaction are unclear. We found that soluble αvβ3 bound to immobilized VEGF165-HBD protein in a dose-dependent manner, but not to the N-terminal domain (NTD) in an ELISA-type binding assay (FIG. 1a,b). This binding was suppressed by heat treatment of the HBD, suggesting that the interaction requires proper folding of the HBD. This binding was inhibited by function blocking mAb 7E3 specific to β3 integrin (FIG. 1c), which is mapped in the classical ligand-binding site within the β3 integrin subunit [25, 26]. Binding of soluble αvβ3 to the HBD is cation dependent (Mn²⁺>Mg²⁺=Ca²⁺>EDTA) (FIG. 1d). In a surface plasmon resonance study, the K_{D} of HBD-αvβ3 interaction was calculated as 4.7×10⁻⁷ M (FIG. 1e). These results indicate that the VEGF165 HBD is a specific ligand for αvβ3 (FIG. 1f). These findings predict that the VEGF165 binds to αvβ3 through the HBD and to KDR via the N-terminal domains (NTD), resulting in the integrin-VEGF165-KDR ternary complex.

Mapping Amino Acid Residues within the VEGF165 HBD for Integrin Binding

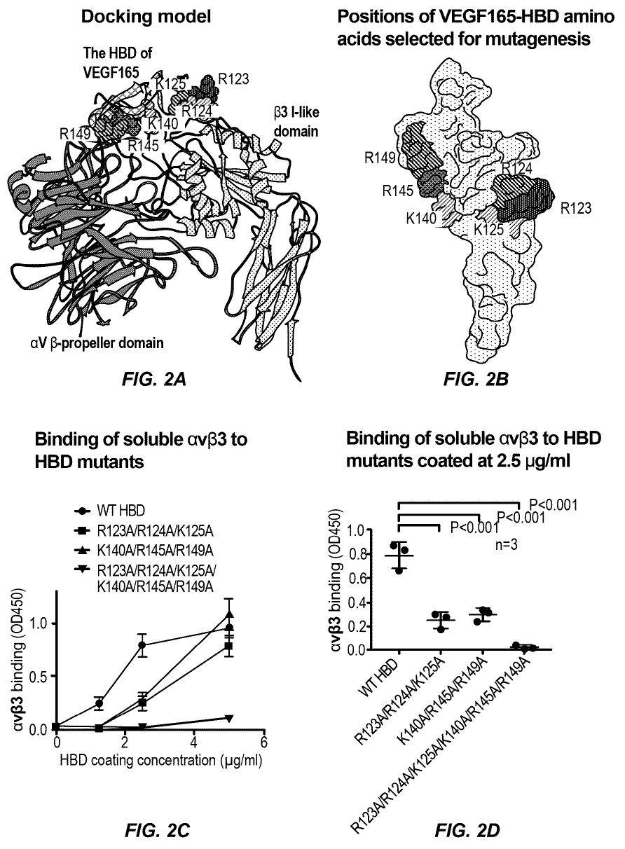

To predict which amino acid residues are involved in αvβ3 binding, we performed docking simulations between the HBD (2VGH.pdb) and αvβ3 (1L5G.pdb, with open headpiece) using Autodock3 (FIG. 2a). The simulation predicted that the HBD binds to αvβ3 at high affinity (docking energy at −24.6 Kcal/mol). We selected several Arg or Lys residues of the HBD in the predicted binding interface for mutagenesis (FIG. 2b). Amino acid residues predicted as involved in the HBD-αvβ3 interaction are Arg-123, Arg-124, Lys-125, Lys-140, Arg-145, and Arg-149. Binding of soluble αvβ3 to immobilized HBD wild type vs mutant protein was measured by ELISA-type binding assays. The Arg123/Arg124/Lys125 to Ala (R123A/R124A/K125A) mutation and the Lys140/Arg145/Arg149 to Ala mutation (K140A/R145A/R149A) partially suppressed the binding of αvβ3 to wild type HBD (FIG. 2c). The combined R123A/R124A/K125A/K140A/R145A/R149A mutation nearly completely blocked integrin binding (FIG. 2d). These results indicate that these amino acid residues are critical for VEGF165 HBD binding to αvβ3.

Full-Length VEGF165 with a Mutated HBD Defective in αvβ3 Binding is Defective in Signaling Functions while Still Binding KDR

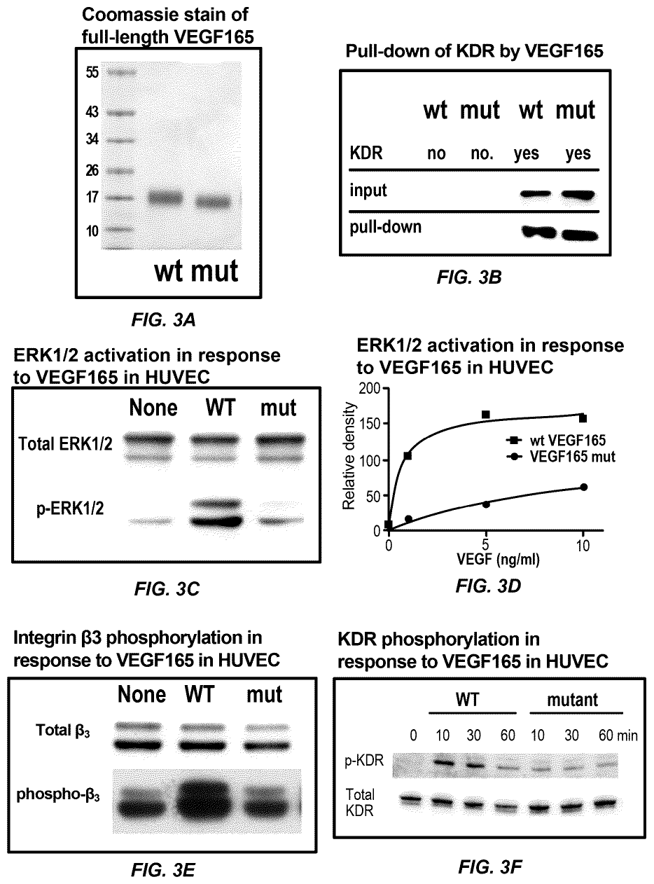

Full-length VEGF165 protein with the combined HBD R123A/R124A/K125A/K140A/R145A/R149A mutations (referred to as VEGF165-HBD mutant) (FIG. 3a) was synthesized in E. coli as insoluble protein, purified under denaturation, refolded and further purified by FPLC gel-filtration. The protein migrated as a single band with expected size (FIG. 3a). The protein bound to immobilized KDR in pull-down assays (FIG. 3b), indicating that KDR binding specificity was retained. The VEGF165-HBD mutant lacked integrin binding αvβ3—as expected. Used as a ligand in human endothelial cell (HUVEC) cultures, VEGF165-HBD mutant protein failed to induce ERK1/2 activation and integrin β3 phosphorylation (FIG. 3c-e). Furthermore, VEGF165-HBD mutant protein also failed to induce KDR Y-1175 phosphorylation in HUVECs (FIG. 3f), despite the ability of the mutant protein to bind KDR (FIG. 3b). These findings indicate that binding of integrin αvβ3 to the HBD of VEGF165 is critical for signaling functions of VEGF165.

Our previous studies showed that mutants of growth factors (e.g., FGF1, IGF1, and fractalkine) that lack integrin binding, still bound to their cognate receptors and can act as dominant-negative antagonists [8-11, 13, 14, 16]. We therefore hypothesize that the VEGF165-HBD mutant defective in integrin binding can act as an antagonist for KDR-VEGF165 mediated cell responses and reveal target information for novel synergistic therapeutic approaches.

### Discussion

The present study establishes that integrin αvβ3 binds to the isolated HBD, not NTD, of VEGF165, indicating that the HBD is a specific ligand for αvβ3. This is consistent with a previous report that the HBD contains an αvβ3-binding site [24]. The proteolytic removal of the HBD markedly reduced the mitogenicity of VEGF165, suggesting that the binding of the NTD to KDR D2D3 may not be sufficient for potent mitogenicity [21, 27]. The present results suggest that VEGF165 bind to integrin αvβ3 via the HBD and to KDR via NTD and as a result induces integrin-VEGF165-KDR ternary complex formation on the cell surface.

The present study identified amino acid residues in the HBD that are critical for integrin binding (Arg-123, Arg-124, Lys-125, Lys-140, Arg-145, and Arg-149) using docking simulation and mutagenesis. Mutations R123A/R124A/K125A and K140A/R145A/R149A effectively reduced integrin binding. Combined mutations of all these amino acids (R123A/R124A/K125A/K140A/R145A/R149A) almost completely suppressed the binding of αvβ3 to the HBD. We generated full-length VEGF165 with the combined HBD mutations (R123A/R124A/K125A/K140A/R145A/R149A) did not affect KDR binding, but effectively suppressed ERK1/2 activation, phosphorylation of integrin 03, and KDR phosphorylation in HUVEC. These findings suggest that the binding of VEGF165 to αvβ3 through the HBD plays a critical role in VEGF165 signaling. We propose that the ternary complex model can be applied to VEGF165.

We have reported that FGF1 and FGF2 directly bind to integrins and these interactions lead to ternary complex formation (integrin-FGF-FGFR), which is required for their signaling functions (the ternary complex model) [8-11]. This indicates that direct binding of integrins to FGF that positively regulates FGF signaling is required. Consistently, mutants of FGF defective in integrin binding are functionally defective and suppress signaling induced by WT FGF (dominant-negative antagonists) [9, 10]. These findings are consistent with the reports that antagonists to integrins (e.g., αvβ3) block angiogenesis induced by FGF2 [4]. We have also reported that integrins also directly bind to several growth factors other than FGF (IGF-1 and -2, neuregulin-1, fractalkine, and CD40L) and positively regulate their signaling functions [12-16, 28]. We showed that growth factor mutants defective in integrin binding (e.g., IGF1, IGF2, fractalkine and CD40L) acted as dominant-negative antagonists [13, 14, 28]. Thus, the VEGF165 mutant (R123A/R124A/K125A/K140A/R145A/R149A) acts as a dominant-negative antagonist.

TABLE 1
Amino acid residues predicted to be involved in HBD binding
to integrin αvβ3. Amino acid residues within 0.6 nm between
the HBD and αvβ3 were selected using Swiss PDB Viewer
(version 4.1) (Swiss Institute of Bioinformatics, Basel, Swiss).

| HBD | αv | β3 |
|---|---|---|
| Glu122, Arg123, Arg124, | Ala149, Asp150, | Tyr122, Ser123, |
| Lys 125, His126, Leu127, | Gly151, Phe177, | Met124, Lys125, |
| Phe128, Val129, Gln130, | Tyr178, Gln180, | Asp126, Asp127, |
| Asp131, Pro132, Gln133, | Arg211, Thr212, | Met180, Lys181, |
| Lys140, Ser144, Arg145, | Ala213, Gln214, | Thr182, Thr183, |
| Cys146, Ala148, Arg149, | Ala215, Asp218 | Arg214, Asn215, |
| Gln150, Leu151, Cys160 | | Asp251, Thr334, |
| | | Met335, Asp336, |
| | | Ser337 |

### REFERENCES

1. Hynes, R. O., Integrins: bidirectional, allosteric signaling machines. Cell, 2002. 110(6): p. 673-87.
2. Giancotti, F. G. and E. Ruoslahti, Integrin signaling. Science, 1999. 285(5430): p. 1028-32.
3. Takada, Y., X. Ye, and S. Simon, The integrins. Genome Biol, 2007. 8(5): p. 215. 10.1186/gb-2007-8-5-215 PMC1929136
4. Brooks, P. C., R. A. Clark, and D. A. Cheresh, Requirement of vascular integrin alpha v beta 3for angiogenesis. Science, 1994. 264(5158): p. 569-71.
5. Kim, S. H., J. Turnbull, and S. Guimond, Extracellular matrix and cell signalling: the dynamic cooperation of integrin, proteoglycan and growth factor receptor. J Endocrinol, 2011. 209(2): p. 139-51. 10.1530/JOE-10-0377
6. Odenthal, J., R. Takes, and P. Friedl, Plasticity oftumor cell invasion: governance by growthfactors and cytokines. Carcinogenesis, 2016. 37(12): p. 1117-1128. 10.1093/carcin/bgw098
7. Desgrosellier, J. S. and D. A. Cheresh, Integrins in cancer: biological implications and therapeutic opportunities. Nat Rev Cancer, 2010. 10(1): p. 9-22. 10.1038/nrc2748 PMC4383089
8. Mori, S., C. Y. Wu, S. Yamaji, J. Saegusa, B. Shi, Z. Ma, Y. Kuwabara, K. S. Lam, R. R. Isseroff, Y. K. Takada, and Y. Takada, Direct binding of integrin alphavbeta3 to FGF1 plays a role in FGF1 signaling. J Biol Chem, 2008. 283(26): p. 18066-75. 10.1074/jbc.M801213200 PMC2440593
9. Yamaji, S., J. Saegusa, K. Ieguchi, M. Fujita, S. Mori, Y. K. Takada, and Y. Takada, A novel fibroblast growth factor-1 (FGF1) mutant that acts as an FGF antagonist. PLoS One, 2010. 5(4): p. e10273. 10.1371/journal.pone.0010273 PMC2858075
10. Mori, S., V. Tran, K. Nishikawa, T. Kaneda, Y. Hamada, N. Kawaguchi, M. Fujita, J. Saegusa, Y. K. Takada, N. Matsuura, M. Zhao, and Y. Takada, A dominant-negative FGF1 mutant (the R50E mutant) suppresses tumorigenesis and angiogenesis. PLoS One, 2013. 8(2): p. e57927. 10.1371/journal.pone.0057927 PMC3585250
11. Mori, S., N. Hatori, N. Kawaguchi, Y. Hamada, T. C. Shih, C. Y. Wu, K. S. Lam, N. Matsuura, H. Yamamoto, Y. K. Takada, and Y. Takada, The integrin-binding defective FGF2 mutants potently suppress FGF2 signaling and angiogenesis. Biosci Rep, 2017. 37(2). 10.1042/BSR20170173 PMC5482197
12. Saegusa, J., S. Yamaji, K. Ieguchi, C. Y. Wu, K. S. Lam, F. T. Liu, Y. K. Takada, and Y. Takada, The direct binding of insulin-like growth factor-1 (IGF-1) to integrin alphavbeta3 is involved in IGF-1 signaling. J Biol Chem, 2009. 284(36): p. 24106-14. 10.1074/jbc.M109.013201 PMC2782004
13. Fujita, M., Y. K. Takada, and Y. Takada, Integrins alphavbeta3 and alpha4beta1 act as coreceptors for fractalkine, and the integrin-binding defective mutant offractalkine is an antagonist of CX3CR1. J Immunol, 2012. 189(12): p. 5809-19. 10.4049/jimmunol.1200889 PMC3518660
14. Cedano Prieto, D. M., Y. Cheng, C. C. Chang, J. Yu, Y. K. Takada, and Y. Takada, Direct integrin binding to insulin-like growth factor-2 through the C-domain is required for insulin-like growthfactor receptor type 1 (IGFIR) signaling. PLoS One, 2017. 12(9): p. e0184285. 10.1371/journal.pone.0184285 PMC5584928
15. Ieguchi, K., M. Fujita, Z. Ma, P. Davari, Y. Taniguchi, K. Sekiguchi, B. Wang, Y. K. Takada, and Y. Takada, Direct binding of the EGF-like domain of neuregulin-1 to integrins ({alpha}v{beta}3 and {alpha}6{beta}4) is involved in neuregulin-1/ErbB signaling. J Biol Chem, 2010. 285(41): p. 31388-98. 10.1074/jbc.M110.113878 PMC2951213
16. Takada, Y. K., J. Yu, M. Shimoda, and Y. Takada, Integrin Binding to the Trimeric Interface of CD40L Plays a Critical Role in CD40/CD40L Signaling. J Immunol, 2019. 203(5): p. 1383-1391. 10.4049/jimmunol.1801630
17. Ferrara, N., Vascular endothelial growth factor: basic science and clinical progress. Endocr Rev, 2004. 25(4): p. 581-611. 10.1210/er.2003-0027
18. Ferrara, N., K. Carver-Moore, H. Chen, M. Dowd, L. Lu, K. S. O'Shea, L. Powell-Braxton, K. J. Hillan, and M. W. Moore, Heterozygous embryonic lethality induced by targeted inactivation of the VEGF gene. Nature, 1996. 380(6573): p. 439-42. 10.1038/380439a0
19. Waltenberger, J., L. Claesson-Welsh, A. Siegbahn, M. Shibuya, and C. H. Heldin, Different signal transduction properties of KDR and Fltl, two receptors for vascular endothelial growth factor J Biol Chem, 1994. 269(43): p. 26988-95.
20. Gille, H., J. Kowalski, B. Li, J. LeCouter, B. Moffat, T. F. Zioncheck, N. Pelletier, and N. Ferrara, Analysis of biological effects and signaling properties of Flt-1 (VEGFR-1) and KDR (VEGFR-2). A reassessment using novel receptor-specific vascular endothelial growth factor mutants. J Biol Chem, 2001. 276(5): p. 3222-30.
21. Keyt, B. A., L. T. Berleau, H. V. Nguyen, H. Chen, H. Heinsohn, R. Vandlen, and N. Ferrara, The carboxyl-terminal domain (111-165) of vascular endothelial growth factor is critical for its mitogenic potency. J Biol Chem, 1996. 271(13): p. 7788-95.
22. Takagi, J., H. P. Erickson, and T. A. Springer, C-terminal opening mimics ‘inside-out’ activation of integrin alpha5beta1. Nat Struct Biol, 2001. 8(5): p. 412-6. 10.1038/87569 87569 [pii]
23. Saegusa, J., N. Akakura, C. Y. Wu, C. Hoogland, Z. Ma, K. S. Lam, F. T. Liu, Y. K. Takada, and Y. Takada, Pro-inflammatory secretory phospholipase A2 type IIA binds to integrins alphavbeta3 and alpha4beta1 and induces proliferation of monocytic cells in an integrin-dependent manner. J Biol Chem, 2008. 283(38): p. 26107-15. 10.1074/jbc.M804835200 PMC2533795
24. Hutchings, H., N. Ortega, and J. Plouet, Extracellular matrix-bound vascular endothelial growth factor promotes endothelial cell adhesion, migration, and survival through integrin ligation. FASEB J, 2003. 17(11): p. 1520-2. 10.1096/fj.02-0691fje
25. Puzon-McLaughlin, W., T. Kamata, and Y. Takada, Multiple discontinuous ligand-mimetic antibody binding sites define a ligand binding pocket in integrin alpha(IIb)beta(3). J Biol Chem, 2000. 275(11): p. 7795-802. 10.1074/jbc.275.11.7795
26. Artoni, A., J. Li, B. Mitchell, J. Ruan, J. Takagi, T. A. Springer, D. L. French, and B. S. Coller, Integrin beta3 regions controlling binding of murine mAb 7E3: implications for the mechanism of integrin alphaII beta3 activation. Proc Natl Acad Sci USA, 2004. 101(36): p. 13114-20. 10.1073/pnas.0404201101 0404201101 [pii]
27. Plouet, J., F. Moro, S. Bertagnolli, N. Coldeboeuf, H. Mazarguil, S. Clamens, and F. Bayard, Extracellular cleavage of the vascular endothelial growth factor 189-amino acid form by urokinase is required for its mitogenic effect. J Biol Chem, 1997. 272(20): p. 13390-6. 10.1074/jbc.272.20.13390
28. Fujita, M., K. Ieguchi, D. M. Cedano-Prieto, A. Fong, C. Wilkerson, J. Q. Chen, M. Wu, S. H. Lo, A. T. Cheung, M. D. Wilson, R. D. Cardiff, A. D. Borowsky, Y. K. Takada, and Y. Takada, An integrin binding-defective mutant of insulin-like growth factor-1 (R36E/R37E IGF1) acts as a dominant-negative antagonist of the IGF1 receptor (IGF1R) and suppresses tumorigenesis but still binds to IGF1R. J Biol Chem, 2013. 288(27): p. 19593-603. 10.1074/jbc.M113.470872 PMC3707660

### Example 2

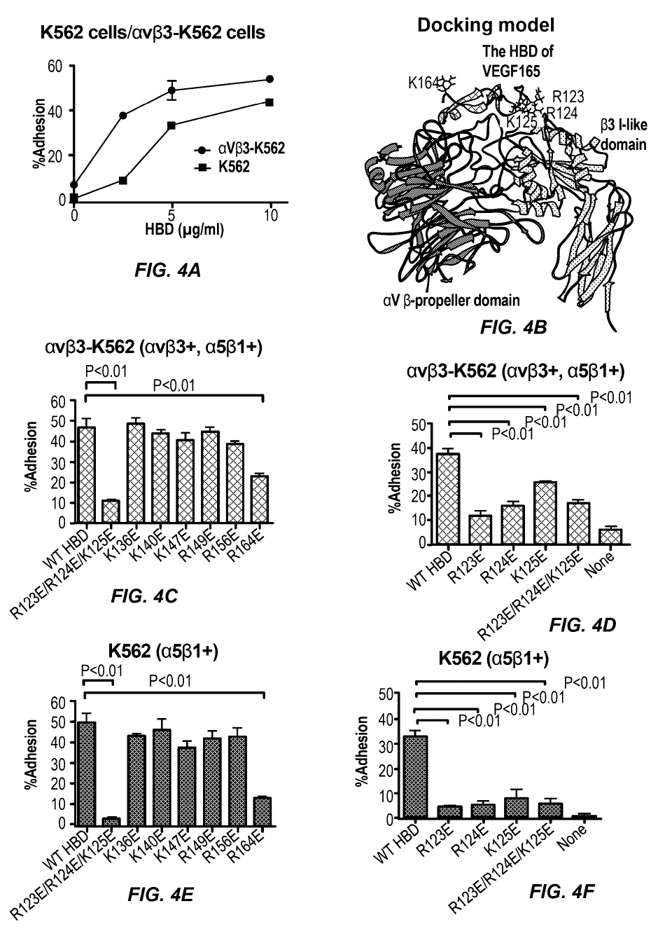

We performed docking simulation of the interaction between αvβ3 and the heparin-binding domain to predict how the αvβ3 binds to the heparin-binding domain. The docking model predicted several amino acid residues within the heparin-binding domain that are critical for αvβ3 binding. Mutation of these residues to Ala suppressed the integrin binding. We found that combined mutation of Arginine or Lysine residues at positions 123, 124, and 125 to Glu (R123E/R124E/K125E) also efficiently suppressed integrin binding to the heparin-binding domain in adhesion assays using αvβ3-K562 cells. See FIG. 4. Mutation of individual Arg123, Arg124, and Lys125 residues to Glu at fewer than all three positions also suppressed the binding, indicating that the reduced integrin binding to the combined mutation is not due to global conformational changes by the mutations. These findings indicate that R123E/R123E/K125E mutation is also effective in blocking integrin binding to VEGF165. In addition, we found that the heparin-binding domain supported adhesion of parent K562 cells that only express integrin α5β1 and the R123E/R124E/K125E mutation effectively suppressed adhesion of K562 cells, indicating that integrin α5β1 binds to the heparin-binding domain as a manner similar to that of αvβ3.

All patents, patent applications, and other publications, including GenBank Accession Numbers, cited in this application are incorporated by reference in the entirety for all purposes.

1. An isolated polypeptide comprising SEQ ID NO:1 with at least one mutation in the 111-165 segment, wherein the polypeptide has decreased binding to integrin αvβ3 compared with SEQ ID NO:1.

2. The polypeptide of claim 1, comprising at least one substitution at residue 123, 124, 125, 140, 145, or 149.

3. The polypeptide of claim 1, wherein the at least one substitution is an A or E substitution.

4. The polypeptide of claim 1, comprising SEQ ID NO:2 and a heterologous peptide.

5. The polypeptide of claim 1, consisting of SEQ ID NO:2.

6. A nucleic acid comprising a polynucleotide sequence encoding the polypeptide of claim 1.

7. An expression cassette comprising a polynucleotide sequence encoding the peptide of claim 1 operably linked to a promoter.

8. A vector comprising the expression cassette of claim 7.

9. A host cell comprising the expression cassette of claim 7.

10. A composition comprising (1) the polypeptide of claim 1; and (2) a pharmaceutically acceptable excipient.

11. A method for treating cancer or inflammation, comprising administering to a subject in need thereof an effective amount of the composition of claim 10.

12. The method of claim 11, wherein the composition is administered to the subject by oral, intravenous, intraperitoneal, intraosseous, intramuscular, or subcutaneous administration.

13. The method of claim 11, wherein the subject is suffering from or at risk of developing a condition involving inappropriate angiogenesis.

14. The method of claim 13, wherein the subject is suffering from or at risk of developing cancer.

15. A method for identifying an inhibitor of VEGF-integrin αvβ3 binding, comprising the steps of
(1) contacting integrin αvβ3 and a polypeptide comprising the 111-165 segment of SEQ ID NO:1, in the presence of a test compound, under conditions permissible for VEGF-integrin αvβ3 binding; and
(2) detecting the level of polypeptide-integrin αvβ3 binding, wherein a decrease in the level of binding when compared with the level of binding in the absence of the test compound indicates the compound as an inhibitor of VEGF-integrin αvβ3 binding.

16. The method of claim 15, wherein the polypeptide consists of SEQ ID NO:1 or the 111-165 segment of SEQ ID NO:1.

17. The method of claim 15, wherein the polypeptide comprises (1) SEQ ID NO:1 or the 111-165 segment of SEQ ID NO:1; and (2) a heterologous amino acid sequence.

18. The method of claim 17, wherein the heterologous amino acid sequence is 6× His.

19. The method of claim 15, wherein the integrin αvβ3 is expressed on a cell surface.

20. A kit for inhibiting angiogenesis or inflammation, comprising a first container containing the composition of claim 10 and a second container containing a second anti-angiogenesis therapeutic agent or a second anti-inflammatory therapeutic agent.
